(19) United States
(12) Patent Application Publication
(10) Pub. No.: US 2009/0202544 A1
(43) Pub. Date: Aug. 13, 2009

(54) Methods of Treating Diseases by Targeting Silt3

(75) Inventors: Nicole Suciu-Foca, New York, NY (US); George Vlad, Forest Hills, NY (US); Raffaello Cortesini, New York, NY (US)

Correspondence Address: ROPES & GRAY LLP, PATENT DOCKETING 39/361, 1211 AVENUE OF THE AMERICAS, NEW YORK, NY, 10036-8704, US

(73) Assignee: The Trustees of Columbia University in the City of New York, New York (US)

(21) Appl. No.: 12/223,496

(22) PCT Filed: Feb. 2, 2007
(86) PCT No.: PCT/US07/03020

§ 371 Date: Feb. 5, 2009

Related U.S. Application Data
(60) Provisional application No. 60/765,406, filed on Feb. 2, 2006.
(60) Provisional application No. 60/852,743, filed on Oct. 18, 2006.

(51) Int. Cl. A61K 39/395; A61K 35/14; C12Q 1/68; A61K 31/7088; C07H 21/04

(52) U.S. Cl. 424/138.1; 424/93.73; 424/159.1; 435/6; 536/23.1

(57) ABSTRACT

This invention provides a method of treating a subject afflicted with a disease, comprising withdrawing blood from the subject, treating the withdrawn blood so as to remove sILT3 from the blood, and returning the treated blood to the subject, thereby treating the subject afflicted with the disease. This invention also provides the above method, further comprising administering an anti-ILT3 antibody to the subject. The invention also provides a method of treating a subject afflicted with a disease, comprising administering to the subject an anti-ILT3 antibody, thereby treating the subject. In one embodiment, the disease is chronic viral disease. In another embodiment, the disease is cancer.

### CROSS-REFERENCE TO RELATED APPLICATIONS

This application claims benefit from U.S. Provisional Application Nos. 60/765,406 filed Feb. 2, 2006 and 60/852,743 filed Oct. 18, 2006, the disclosures of which are hereby incorporated herein in their entirety.

The invention disclosed herein was made in part with government support from the National Institutes of Health under grant nos. AI25210-19 and AI55234-03 and AI55234-04. Accordingly, the U.S. Government has certain rights in this invention.

Throughout this invention, various publications are referenced. Full citations for these publications are presented immediately before the claims. Disclosures of these publications in their entireties are hereby incorporated by reference into this application in order to more fully describe the state of the art to which this invention pertains.

### BACKGROUND OF THE INVENTION

The development of new strategies to promote immune responses in certain viral diseases and malignancies is critical to overcome the limited efficacy of conventional therapies. Research in this area has been fueled by the discovery that regulatory T cells and tolerogenic Antigen Presenting Cells (APC) modulate the immune response to self and non-self antigens. The concept has emerged that bi-directional interactions between APC and antigen experienced T cells can initiate either a tolerogenic or an immunogenic pathway.

There is increasing evidence that the immune response can be inhibited by various CD4⁺ and CD8⁺ regulatory T (T_{R}) cells, which participate in innate and adaptive immunity^{3,4}.

After T cell receptor (TCR) triggering, natural T_{R} cells inhibit immune responses in an antigen-nonspecific, APC-independent and MHC-nonrestricted manner. Natural T_{R}, are anergic, do not produce cytokines and suppress effector T cells by cell-to-cell contact.

Adaptive CD4⁺ and CD8⁺ T_{R} cells are antigen induced, develop in the periphery, and exert their function either by secreting inhibitory cytokines (such as IL-10 and TGF-β) or tolerizing directly the APC with which they interact. Tolerogenic APCs express high levels of inhibitory receptors such as immunoglobulin like transcript (ILT)3, induce T cell anergy and elicit the differentiation of antigen-specific CD4 and CD8 regulatory T cells.

LIRs: Leukocyte Ig-like receptors (“LIRs”) are a family of immunoreceptors expressed predominantly on monocytes and B cells and at lower levels on dendritic cells and natural killer (“NK”) cells. Activation of various immune cell types can be prevented by negative signaling receptors through interactions with specific ligands, such as MHC class I molecules by NK cells. All of the LIR inhibitory receptors, members of subfamily B, contain a cytoplasmic immunoreceptor tyrosine-based inhibitory motif (“ITIM”). Upon MHC class I (or other ligand) engagement and tyrosine phosphorylation of the ITIM, intracellular protein-tyrosine phosphatases such as SHPI are recruited, and an inhibitory signal cascade ensues. Other LIR receptors, members of subfamily A, with short cytoplasmic regions containing no ITIMs and with transmembrane regions containing a charged arginine residue, may initiate stimulatory cascades. One member of subfamily A lacks a transmembrane region and is presumed to be a soluble receptor (1). LIR-5, one type of LIR, is also known as ILT3.

ILT3 Fusion Proteins: A soluble fusion protein made of a soluble portion of ILT3 and the Fc portion of IgG1 is known. However, this fusion protein was used merely as a negative control in an endotoxemia study, and its potential use as a therapeutic was not disclosed (2).

Soluble ILT4: LIR-2, also known as ILT4, is an inhibitory receptor. However, its soluble form was shown to completely restore the proliferation of T-cells activated with LPS and IL-10-treated dendritic cells (3).

### SUMMARY OF THE INVENTION

This invention provides a method of treating a subject afflicted with a disease, comprising withdrawing blood from the subject, removing sILT3 from the withdrawn blood, and returning the sILT3-depleted blood to the subject, thereby treating the subject afflicted with the disease.

This invention provides a method of treating a subject afflicted with a disease, comprising: (A) withdrawing blood from the subject, removing sILT3 from the withdrawn blood, and returning the sILT3-depleted blood to the subject, and (B) administering an anti-ILT3 antibody to the subject, thereby treating the subject afflicted with the disease. This invention further provides the above method, but wherein step (B) is performed prior to or concurrently with step (A).

This invention provides a method of treating a subject afflicted with a disease, comprising administering to the subject an anti-ILT3 antibody, thereby treating the subject.

This invention provides a method of enhancing a subject's ability to mount an immune response, comprising withdrawing blood from the subject, removing sILT3 from the withdrawn blood, and returning the sILT3-depleted blood to the subject, thereby enhancing the subject's ability to mount an immune response.

This invention provides a method of enhancing a subject's ability to mount an immune response, comprising administering to the subject an anti-ILT3 antibody, thereby enhancing the subject's ability to mount an immune response.

This invention provides a method of enhancing a subject's ability to mount an immune response, (A) withdrawing blood from the subject, removing sILT3 from the withdrawn blood, and returning the sILT3-depleted blood to the subject, and (B) administering an anti-ILT3 antibody to the subject, thereby enhancing the subject's ability to mount an immune response. This invention further provides the above method, but wherein step (B) is performed prior to or concurrently with step (A).

This invention provides a method of enhancing remission of a tumor, comprising withdrawing blood from the subject, removing sILT3 from the withdrawn blood, and returning the sILT3-depleted blood to the subject, thereby enhancing remission of the tumor.

This invention provides a method of enhancing remission of a tumor, comprising administering to the subject an anti-ILT3 antibody, thereby enhancing remission of the tumor.

This invention provides a method of enhancing remission of a tumor, (A) withdrawing blood from the subject, removing sILT3 from the withdrawn blood, and returning the sILT3-depleted blood to the subject, and (B) administering an anti-ILT3 antibody to the subject, thereby enhancing remission of the tumor. This invention further provides the above method, but wherein step (B) is performed prior to or concurrently with step (A).

This invention provides a method of decreasing tolerance to a tumor in a subject afflicted with cancer, comprising withdrawing blood from the subject, removing sILT3 from the withdrawn blood, and returning the sILT3-depleted blood to the subject, decreasing tolerance to the tumor.

This invention provides a method of decreasing tolerance to a tumor in a subject afflicted with cancer, comprising administering to the subject an anti-ILT3 antibody, thereby decreasing tolerance to the tumor.

This invention provides a method of decreasing tolerance to a tumor in a subject afflicted with cancer, (A) withdrawing blood from the subject, removing sILT3 from the withdrawn blood, and returning the sILT3-depleted blood to the subject, and (B) administering an anti-ILT3 antibody to the subject, decreasing tolerance to the tumor. This invention further provides the above method, but wherein step (B) is performed prior to or concurrently with step (A).

In one embodiment of the methods described herein, the disease is viral disease, such as a chronic viral disease. In another embodiment, the disease is cancer.

### BRIEF DESCRIPTION OF THE FIGURES

FIG. 1: Schematic drawing of soluble ILT3 expression vector.

FIG. 2: Western Blot analysis of soluble ILT3 fusion protein.

FIGS. 3A-3B: (A) Soluble ILT3 inhibits T lymphocyte proliferation in vitro. (B) Inhibition is abrogated by anti-ILT3 antibody.

FIGS. 4A-4D: (A) Schematic diagram of MIG retroviral expression vectors of ILT3 and ILT3 delta. (B) Fluorescence histograms of ILT3 expression on the surface of KG1.ILT3 and KG1.ILT3delta. (C) Confirmation of the molecular weight of ILT3delta by Western Blot and determination of inability of ILT3delta molecule to recruit SHP-1 by immunoprecipitation and Western blot. (D) Inhibition of protein tyrosine phosphorylation in KG1.ILT3 but not in KG1.ILT3delta cells by crosslinking anti-HLA-DR and anti-ILT3 mAbs.

FIG. 5A-5C: (A) ILT3 and ILT3 delta molecules inhibit proliferation of CD3+CD25−T cells in 5 day primary MLCs. (B) ILT3 and ILT3delta molecules suppress proliferation of CD4+ T cells primed with KG1 cells in 3 day secondary MLC, which can be reversed by addition of IL-2 or anti-ILT3 mAb. (C) CD8+ T cells primed with KG1, but not with KG1.ILT3 are cytotoxic to KG1 cells at ET ratio of 1 to 1.

FIG. 6: sILT3 suppress proliferation of CD3+CD25−T cells in primary and secondary MLCs.

FIG. 7: sILT3 inhibit generation CD8⁺ cytotoxic T cells.

FIG. 8: Frequency of IFN-γ producing CD8+ T cells from T cells primed with allogeneic APC with sILT3 is higher than CD8+ T cells primed with the same APC without sILT3.

FIG. 9A-9D: (A) CD8+ T cells primed with allogeneic APC in the presence of sILT3, but not in the absence of sILT3 suppress proliferation response of naïve CD3⁺CD25⁻ T cells from the same responder to the original stimulator. (B) CD8⁺ T cells primed with KG1.ILT3, but not KG1 suppress proliferation response of naïve CD3⁺CD25⁻ T cells from the same responder KG1 cells. (C) CD8⁺ T cells primed with allogeneic APC with sILT3, but not without sILT3, or primed with KG1.ILT3 cells, but not with KG1 cells express high FOX3 protein in Western blot analysis. (D) CD8⁺ T cells primed with allogeneic APC with sILT3, but not without sILT3 up-regulate ILT3 expression and down-regulate CD86 expression on immature DC derived from the same stimulator for priming.

FIG. 10: FITC-labeled sILT3 proteins stains allogeneic APC activated CD4⁺ T cells at day 3 of primary MLC culture, but not activated CD8⁺ T cells and naïve CD4⁺ and CD8⁺ T cells.

FIG. 11: Shows the growth of KG1 and KG1.ILT3 in humanized SCID mice, and there effects of sILT3 in KG1 growth.

FIG. 12: Effect of ILT3 on tumor allografts. Soluble and membrane ILT3 induce tolerance to allogeneic tumors in hu-SCID mice. (a) Tumor growth was assessed in 5 groups each consisting of 10 hu-SCID mice transplanted with KG1 and treated with rILT3-Fc (♦, filled diamond), human IgG (⋄, open diamond), not-treated (◯, open circle), transplanted with empty vector transfected KG1.MIG (Δ, open triangle) or ILT3 transfected KG1 (▴, filled triangle). (b) Engraftment of human T cells in blood lymph nodes and spleen of hu-SCID mice. HLA class I⁺ CD3⁻ CD34⁺ tumor metastasis in regional lymph nodes were seen in mice with KG1 and KG1.ILT3 tumors. (c) Immunohistochemical staining of CD4⁺ and CD8⁺ human T cells infiltrating KG1 tumor growing in hu-SCID mice treated with rILT3-Fc. (d) Growth of melanoma and pancreatic carcinoma cell lines in hu-SCID mice treated or not-treated with rILT3-Fc.

FIG. 13. Functional characterization of human T cells in transplanted hu-SCID mice. (a) CD8 and CD4 T cells from lymph nodes and spleen of hu-SCID mice with growing KG1 or KG1.ILT3 tumors and from untreated mice which have rejected KG1 tumors were tested for their capacity to inhibit the MLC response of unprimed CD4 T cells to KG1. (b) IL-2 and IFN-γ was detected by qRT-PCR in lymph nodes draining the transplantation site of rejected but not of tolerated KG1 tumors. Granzyme B and Perforin were expressed at high levels in CD8⁺ T cells from hu-SCID mice which rejected the tumor.

FIG. 14. Detection of ILT3 in human sera. (a) Sera from cancer patients and healthy blood donors were tested for soluble ILT3 by ELISA. (b) T cells from a healthy volunteer were tested for MLC reactivity against irradiated KG1 stimulating cells in cultures containing pooled (ILT3 negative) sera from healthy controls or ILT3 positive sera from individual cancer patients (N=29) in the presence or absence of anti-ILT3 mAb or mouse IgG. (c) CD8⁺ T cells allostimulated in cultures containing ILT3 positive sera inhibited the alloreactivity of autologous, unprimed CD3⁺ T cells in a dose-dependent manner. (d) Immunoprecipitation and Western Blot analysis of ILT3 positive serum prior (lane 4) and after depletion of ILT3 (lane 2) and of an ILT3 negative control serum.

FIG. 15. Immunohistochemical staining of tumors and lymph nodes with anti-CD68 and anti-ILT3 antibodies.

FIG. 16. Identification of alternatively spliced ILT3 in tumor biopsies. Schematic representation of the structure of the ILT3 gene and of the mRNA deletion mutant. RT-PCR analysis of ILT3 mRNA and tumor (T) and normal (N) colon tissue of two patients with colon carcinoma.

FIG. 17. Intracellular staining. Intracellular staining for IL-2 and IFN-γ in hu-SCID mice was observed in lymph nodes draining the transplantation site of rejected (panel A), but not of tolerated KG1 tumors (panel C and D) or non-transplanted mice (panel A). There was no IL-10 and TGF-β induction.

FIG. 18. (a) Nucleic acid sequence of the clone providing evidence for the existence of the alternatively spliced ILT3, lacking exons 5-7. (b) Nucleic acid sequence of full length ILT3, showing the sequences of each of exons 4-9. The sequence of sILT3 lacks exons 5-7.

### DETAILED DESCRIPTION OF THE INVENTION

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present invention are generally performed according to conventional methods well known in the art. Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane Antibodies: A Laboratory Manual Cold Spring Harbor Laboratory Press, Cold Spring Harbor, N.Y. (1990); Principles of Neural Science, 4^{th} ed., Eric R. Kandel, James H. Schwart, Thomas M. Jessell editors. McGraw-Hill/Appleton & Lange: New York, N.Y. (2000). The nomenclatures used in connection with, and the laboratory procedures and techniques of, molecular and cellular neurobiology and medicinal and pharmaceutical chemistry described herein are those well known and commonly used in the art. Standard techniques are used for chemical syntheses, chemical analyses, pharmaceutical preparation, formulation, and delivery, and treatment of patients.

Throughout this specification, the word “comprise” or variations such as “comprises” or “comprising” will be understood to imply the inclusion of a stated integer or group of integers but not the exclusion of any other integer or group of integers.

### DEFINITIONS

“Administering” shall mean delivering in a manner which is effected or performed using any of the various methods and delivery systems known to those skilled in the art. Administering can be performed, for example, topically, intravenously, pericardially, orally, via implant, transmucosally, transdermally, intramuscularly, subcutaneously, intraperitoneally, intrathecally, intralymphatically, intralesionally, or epidurally. Administering can also be performed, for example, once, a plurality of times, and/or over one or more extended periods.

“Agent” shall include, without limitation, an organic or inorganic compound, a nucleic acid, a polypeptide, a lipid, a carbohydrate or a physical stimulus. Agents include, for example, agents which are known with respect to structure and/or function, and those which are not known with respect to structure or function.

“Concurrent” administration of two agents shall mean administration wherein the time period over which the first agent is administered either overlaps with, or is coincident with, the time period over which the second agent is administered. For example, a first and a second agent are concurrently administered if the first agent is administered once per week for four weeks, and the second agent is administered twice per week for the first three of those four weeks. Likewise, for example, a first and second agent are concurrently administered if the first and second agent are each administered, in the same or separate pills, on the same day, once per week for four weeks.

“Expression vector” shall mean a nucleic acid encoding a nucleic acid of interest and/or a protein of interest, which nucleic acid, when placed in a cell, permits the expression of the nucleic acid or protein of interest. Expression vectors are well known in the art.

“Extracellular domain of ILT3” shall mean the N-terminal 258 amino acid residues of ILT3 (e.g., human ILT3 having the sequence of GenBank Accession No. U82979), and may also be referred to as sILT3 (or soluble ILT3). A “portion” of the extracellular domain of ILT3 includes, for example, the IgG1-like domain 1 (residues 42-102 of human ILT3), the IgG1-like domain 2 (residues 137-197 of human ILT3), and the N-terminal 250, 240, 230, 220, 210, 200, 190, 180, 170, 160 or 150 amino acid residues of ILT3.

“Function-enhancing mutation”, with respect to the second polypeptide of this invention, shall mean any mutation which confers a physical property (e.g., reduced binding of the Fc moiety to an Fc receptor) to the polypeptide which permits it to better accomplish its therapeutic role (e.g., through increasing its half-life or reducing adverse effects otherwise caused by a subject's immune system).

“ILT3” shall mean “Immunoglobulin-Like Transcript-3”, and is synonymous with “ILT-3”, “LIR-5”, “CD85K” and “LILRB4.” The mRNA coding sequence for human ILT3 is provided under GenBank No. U82979.

“Immunoglobulin” and “antibody” are used synonymously herein, and shall include, by way of example, both naturally occurring and non-naturally occurring antibodies. Specifically, this term includes polyclonal and monoclonal antibodies, and antigen-binding fragments (e.g., Fab fragments, as opposed to Fc fragments) thereof. Furthermore, this term includes chimeric antibodies (e.g., humanized antibodies) and wholly synthetic antibodies, and antigen-binding fragments thereof. Within the scope of the term “antibody” are also antibodies that have been modified in sequence, but remain capable of specific binding to an antigen. Example of modified antibodies are interspecies chimeric and humanized antibodies; antibody fusions; and heteromeric antibody complexes, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513), the disclosure of which is incorporated herein by reference in its entirety).

“Inhibiting” the onset of a disorder shall mean either lessening the likelihood of the disorder's onset, or preventing the onset of the disorder entirely. In the preferred embodiment, inhibiting the onset of a disorder means preventing its onset entirely.

“Mammalian cell” shall mean any mammalian cell. Mammalian cells include, without limitation, cells which are normal, abnormal and transformed, and are exemplified by neurons, epithelial cells, muscle cells, blood cells, immune cells, stem cells, osteocytes, endothelial cells and blast cells.

“Nucleic acid” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

“Operably affixed”, with respect to the second polypeptide of this invention, shall mean affixed (e.g., via peptide bond) in a manner permitting the ILT3 moiety thereof to inhibit the proliferation of CD4⁺ T cells. In one embodiment, a polypeptide linker of 10, 11, 12, 13, 14, 15 or 16 amino acid residues in length is used to join the ILT3 and Fc moieties.

“Pharmaceutically acceptable carriers” are well known to those skilled in the art and include, but are not limited to, 0.01-0.1 M and preferably 0.05 M phosphate buffer or 0.8% saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Porphyrin or Lipofectin may also be used as a delivery agent. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like.

“Polypeptide” and “protein” are used interchangeably herein, and each means a polymer of amino acid residues. The amino acid residues can be naturally occurring or chemical analogues thereof. Polypeptides and proteins can also include modifications such as glycosylation, lipid attachment, sulfation, hydroxylation, and ADP-ribosylation. A variety of methods for labeling polypeptides and substituents or labels useful for such purposes are well known in the art, and include radioactive isotopes such as ¹²⁵I, ³²P, ³⁵S, and ³H, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods for labeling polypeptides are well known in the art. See, e.g., Ausubel et al., 1992, hereby incorporated by reference.

“Prophylactically effective amount” means an amount sufficient to inhibit the onset of a disorder or a complication associated with a disorder in a subject.

“Subject” shall mean any organism including, without limitation, a mammal such as a mouse, a rat, a dog, a guinea pig, a ferret, a rabbit and a primate. In the preferred embodiment, the subject is a human being.

“Therapeutically effective amount” means any amount of an agent which, when administered to a subject afflicted with a disorder against which the agent is effective, causes the subject to be treated.

“Transplant rejection” shall mean the adverse response by the immune system of a subject who has received a transplant (e.g., of an organ or tissue). Transplanted organs in this context include, for example, heart, kidney, skin, lung, liver, eye and bone. Transplanted tissue in this context includes, for example, vascular tissue.

“Treating” a subject afflicted with a disorder shall mean causing the subject to experience a reduction, remission or regression of the disorder and/or its symptoms. In one embodiment, recurrence of the disorder and/or its symptoms is prevented. In the preferred embodiment, the subject is cured of the disorder and/or its symptoms.

### DESCRIPTION

This invention provides a method of treating a subject afflicted with a disease, comprising withdrawing blood from the subject, removing sILT3 from the withdrawn blood, and returning the sILT3-depleted blood to the subject, thereby treating the subject afflicted with the disease. In one embodiment, the disease is a viral disease, such as a chronic viral disease. In another embodiment, the disease is cancer. In yet another embodiment the disease is characterized by increased levels of sILT3 in blood.

The subject treated by any of the methods of the invention may be, without limitation, a human, mammal, including non-human mammal, dog, cat, horse, cow, sheep, chicken.

In one embodiment of the invention, the disease is cancer, and the treatment results in cancer or tumor remission. As used herein, “remission” refers to the shrinkage or reduction of the cancer or tumor. Remission includes complete remission, such that the cancer or malignancy or tumor is no longer evidenced in the subject, and partial remission, wherein the cancer or malignancy or tumor is reduced but is still present in the subject. Accordingly, as used herein, remission may contemplate any amount of reduction of the cancer or tumor, so long as there is a reduction in the cancer or tumor after a treatment described herein.

“Therapeutically effective amount” refers to that amount of the therapeutic agent being administered which will relieve to some extent one or more of the symptoms of the disorder being treated. In reference to the treatment of cancer, a therapeutically effective amount refers to that amount which has at least one of the following effects: reducing the size of the tumor; inhibiting (that is, slowing to some extent, preferably stopping) tumor metastasis; inhibiting to some extent (that is, slowing to some extent, preferably stopping) tumor growth, and relieving to some extent (or, preferably, eliminating) one or more symptoms associated with the cancer.

“Treat”, “treating” and “treatment” refer to a method of alleviating or abrogating a biological disorder and/or its attendant symptoms. With regard to cancer, these terms simply mean that the life expectancy of an individual affected with a cancer will be increased or that one or more of the symptoms of the disease will be reduced.

The cancer may be any cancer including, but not limited to, the following: AIDS related cancer, adrencortical cancer, basal cell carcinoma, bladder cancer, bowel cancer, brain or CNS tumor, breast cancer, carcinoid tumor, cervical cancer, chondrosarcoma, choriocarcinoma, colon cancer, colorectal cancer, endocrine cancer, endometrial cancer, esophageal cancer, Ewing's Sarcoma, eye cancer, gastric cancer, gastrointestinal cancer, genitourinary cancer, glioma, gynecological cancer, head and neck cancer, hepatocellular cancer, Hodgkin's Disease, hypopharynx cancer, islet cell cancer, Kaposi's sarcoma, kidney cancer, laryngeal cancer, leukemia, liver cancer, lung cancer, hematologic malignancy, lymphoma, (including chronic lymphocytic leukemia, acute lymphocytic leukemia and cutaneous T cell lymphoma), melanoma, mesothelioma, multiple myeloma, myeloid malignancy, nasopharyngeal cancer, neuroblastoma, Non-Hodgkin's lymphoma, non-melanoma skin cancer, oesophageal cancer, osteosarcoma, ovarian cancer, pancreas cancer, pituitary cancer, prostate cancer, renal cell carcinoma, retinoblastoma, rhabdomyosarcoma, sarcoma, skin cancer, squamous cell carcinoma, stomach cancer, testicular cancer, thymus cancer, thyroid cancer, transitional cell cancer, trophoblastic cancer, uterus cancer, vaginal cancer, waldenstrom's macroglobinemia, and Wilms' tumor.

In one embodiment of the invention, the disease is a chronic viral disease. The chronic viral disease includes but is not limited to HIV (Human Immunodeficiency Virus), hepatitis C virus, hepatitis B virus, herpesvirus, Epstein-Barr virus and cytomegalovirus. The subject may also be afflicted with AIDS (Acquired Immune Deficiency Syndrome). In one embodiment, the treatment results in an enhancement of the subject's ability to mount an immune response.

The sILT3 may be removed from the subject's blood by methods known to one skilled in the art. In one embodiment, the sILT3 is removed by plasma exchange (plasmapheresis). An example of plasmapheresis comprises separating the blood's plasma fraction from the blood's cellular fraction. The plasma fraction is then treated so as to remove the sILT3 therefrom. The sILT3 may be either totally depleted or partially depleted. The resulting sILT3 depleted plasma fraction is then recombined with the cellular fraction, and then the recombined blood is returned to the subject.

In one embodiment, the plasma exchange is conducted by employing anti-ILT3 antibodies, or any other protein or compound capable of binding sILT3, to capture the sILT3 and effect removal of the sILT3. The anti-ILT3 antibodies may be coated or otherwise fixed to a surface, such as a solid surface. Examples of solid surfaces include columns, such as a sepharose column, and filters. The plasma fraction is then passed through the solid surface. The techniques as used herein to capture the sILT3 may be adapted from Molecular Absorbent Recirculating System (MARS7) methodology used in patients with terminal liver disease.

This invention provides a method of treating a subject afflicted with a disease, comprising: (A) withdrawing blood from the subject, removing sILT3 from the withdrawn blood, and returning the sILT3-depleted blood to the subject, and (B) administering an anti-ILT3 antibody to the subject, thereby treating the subject afflicted with the disease. This invention further provides the above method, but wherein step (B) is performed prior to or concurrently with step (A). In one embodiment, the disease is a viral disease, such as a chronic viral disease. In another embodiment, the disease is cancer.

This invention provides a method of treating a subject afflicted with a disease, comprising administering to the subject an anti-ILT3 antibody, thereby treating the subject. In one embodiment, the disease is a viral disease, such as a chronic viral disease. In another embodiment, the disease is cancer. In yet another embodiment the diseases is characterized by increased levels of sILT3 in blood.

This invention provides a method of enhancing a subject's ability to mount an immune response, comprising withdrawing blood from the subject, removing sILT3 from the withdrawn blood, and returning the sILT3-depleted blood to the subject, thereby enhancing the subject's ability to mount an immune response. The subject of the methods described herein may be one who is immune compromised. The subject may be one in need of an enhanced immune response.

This invention provides a method of enhancing a subject's ability to mount an immune response, comprising (A) withdrawing blood from the subject, removing sILT3 from the withdrawn blood, and returning the sILT3-depleted blood to the subject, and (B) administering an anti-ILT3 antibody to the subject, thereby enhancing the subject's ability to mount an immune response. This invention further provides the above method, but wherein step (B) is performed prior to or concurrently with step (A).

This invention provides a method of enhancing a subject's ability to mount an immune response, comprising administering to the subject an anti-ILT3 antibody, thereby enhancing the subject's ability to mount an immune response.

This invention provides a method of enhancing remission of a tumor, comprising withdrawing blood from the subject, removing sILT3 from the withdrawn blood, and returning the sILT3-depleted blood to the subject, thereby enhancing remission of the tumor.

This invention provides a method of enhancing remission of a tumor, comprising (A) withdrawing blood from the subject, removing sILT3 from the withdrawn blood, and returning the sILT3-depleted blood to the subject, and (B) administering an anti-ILT3 antibody to the subject, thereby enhancing remission of the tumor. This invention further provides the above method, but wherein step (B) is performed prior to or concurrently with step (A). In one embodiment, the disease is a viral disease, such as a chronic viral disease. In another embodiment, the disease is cancer.

This invention provides a method of enhancing remission of a tumor, comprising administering to the subject an anti-ILT3 antibody, thereby enhancing remission of the tumor.

This invention provides a method of decreasing tolerance to a tumor in a subject afflicted with cancer, comprising withdrawing blood from the subject, removing sILT3 from the withdrawn blood, and returning the sILT3-depleted blood to the subject, thereby decreasing tolerance to the tumor.

This invention provides a method of decreasing tolerance to a tumor in a subject afflicted with cancer, comprising (A) withdrawing blood from the subject, removing sILT3 from the withdrawn blood, and returning the sILT3-depleted blood to the subject, and (B) administering an anti-ILT3 antibody to the subject, thereby decreasing tolerance to the tumor. This invention further provides the above method, but wherein step (B) is performed prior to or concurrently with step (A).

This invention provides a method of decreasing tolerance to a tumor in a subject afflicted with cancer, comprising administering to the subject an anti-ILT3 antibody, thereby decreasing tolerance to the tumor.

In one embodiment of the methods described herein, administration of the anti-ILT3 antibody to the subject results in formation of complexes between the antibody and sILT3 and/or membrane-bound ILT3. Such complexes may be cleared by phagocytic cells, thus reducing the subject's levels of sILT3, such as that present in the serum.

In one embodiment of the methods described herein, the anti-ILT3 antibody is a monoclonal antibody. In one embodiment of the methods described herein, the anti-ILT3 antibody is a humanized antibody. In one embodiment of the methods described herein, the anti-ILT3 antibody is a fully human antibody. The antibodies described herein may be non-cytolitic antibodies.

Various procedures known within the art may be used for the production of polyclonal or monoclonal antibodies directed against a polypeptide of the invention, or against derivatives, fragments, analogs homologs or orthologs thereof. See, for example, ANTIBODIES: A LABORATORY MANUAL, Harlow and Lane (1988) Cold Spring Harbor Laboratory Press, Cold Spring Harbor, N.Y. Some of these antibodies are discussed below.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein (1975) Nature, 256:495. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The antibodies directed against the polypeptide antigens of the invention can further comprise humanized antibodies or human antibodies. These, antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al. (1986) Nature, 321:522-525; Riechmann et al. (1988) Nature, 332:323-327; Verhoeyen et al. (1988) Science, 239:1534-1536), by substituting rodent CDRs or CDR sequences for the corresponding sequences of a human antibody. (See also U.S. Pat. No. 5,225,539.)

Where an “antibody” is referred to herein with respect to the invention, it is normally understood that an antigen-binding portion thereof may also be used. An antigen-binding portion competes with the intact antibody for specific binding. See generally, Fundamental Immunology, Ch. 7 (Paul, W., ed., 2nd ed. Raven Press, N.Y. (1989)) (incorporated by reference in its entirety for all purposes). Antigen-binding portions may be produced by recombinant DNA techniques or by enzymatic or chemical cleavage of intact antibodies. In some embodiments, antigen-binding portions include Fab, Fab′, F(ab′)₂, Fd, Fv, dAb, and complementarity determining region (CDR) fragments, single-chain antibodies (scFv), chimeric antibodies, diabodies and polypeptides that contain at least a portion of an antibody that is sufficient to confer specific antigen binding to the polypeptide. As used herein, a Fd fragment means an antibody fragment that consists of the V_{H} and C_{H}1 domains; an Fv fragment consists of the V_{L} and V_{H} domains of a single arm of an antibody; and a dAb fragment (Ward et al., Nature 341:544-546 (1989)) consists of a V_{H} domain. In some embodiments, the antibody is a single-chain antibody (scFv) in which a V_{L} and V_{H} domains are paired to form a monovalent molecules via a synthetic linker that enables them to be made as a single protein chain. (Bird et al., Science 242:423-426 (1988) and Huston et al., Proc. Natl. Acad. Sci. USA 85:5879-5883 (1988).) In some embodiments, the antibodies are diabodies, i.e., are bivalent antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites. (See e.g., Holliger P. et al., Proc. Natl. Acad. Sci. USA 90:6444-6448 (1993), and Poljak R. J. et al., Structure 2:1121-1123 (1994).) In some embodiments, one or more CDRs from an antibody of the invention may be incorporated into a molecule either covalently or noncovalently to make it an immunoadhesin that specifically binds to c-Met. In such embodiments, the CDR(s) may be incorporated as part of a larger polypeptide chain, may be covalently linked to another polypeptide chain, or may be incorporated noncovalently.

An ILT3 antibody or antigen-binding portion of the invention can be derivatized or linked to another molecule (e.g., another peptide or protein). In general, the antibodies or portion thereof are derivatized such that the ILT3 binding is not affected adversely by the derivatization or labeling. Accordingly, the antibodies and antibody portions of the invention are intended to include both intact and modified forms of the human anti-ILT3 antibodies described herein. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detection agent, a pharmaceutical agent, and/or a protein or peptide that can mediate association of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Another type of derivatized antibody is a labeled antibody. Useful detection agents with which an antibody or antigen-binding portion of the invention may be derivatized include fluorescent compounds, including fluorescein, fluorescein isothiocyanate, rhodamine, 5-dimethylamine-1-napthalenesulfonyl chloride, phycoerythrin, lanthanide phosphors and the like. An antibody can also be labeled with enzymes that are useful for detection, such as horseradish peroxidase, β-galactosidase, luciferase, alkaline phosphatase, glucose oxidase and the like. When an antibody is labeled with a detectable enzyme, it is detected by adding additional reagents that the enzyme uses to produce a reaction product that can be discerned. For example, when the agent horseradish peroxidase is present, the addition of hydrogen peroxide and diaminobenzidine leads to a colored reaction product, which is detectable. An antibody can also be labeled with biotin, and detected through indirect measurement of avidin or streptavidin binding. An antibody can also be labeled with a predetermined polypeptide epitope recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

An anti-ILT3 antibody can also be derivatized with a chemical group such as polyethylene glycol (PEG), a methyl or ethyl group, or a carbohydrate group. These groups are useful to improve the biological characteristics of the antibody, e.g., to increase serum half-life.

The antibodies and antigen-binding portions of the present invention can be incorporated into pharmaceutical compositions suitable for administration to a subject. Typically, the pharmaceutical composition comprises an antibody or antigen-binding portion of the invention and a pharmaceutically acceptable carrier. As used herein, “pharmaceutically acceptable carrier” means any and all solvents, dispersion media, coatings, antibacterial and antifungal agents, isotonic and absorption delaying agents, and the like that are physiologically compatible. Some examples of pharmaceutically acceptable carriers are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody.

The compositions of this invention may be in a variety of forms, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection. Formulations for injection may be presented in unit dosage form, e.g., in ampoules or in multi-dose containers, with or without an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the anti-ILT3 antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibodies or antibody portions of the present invention can be administered by a variety of methods known in the art, although for many therapeutic applications, the preferred route/mode of administration is subcutaneous, intramuscular, or intravenous infusion. As will be appreciated by the skilled artisan, the route and/or mode of administration will vary depending upon the desired results.

In certain embodiments, the antibody compositions of the present invention may be prepared with a carrier that will protect the antibody against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are generally known to those skilled in the art. See, e.g., Sustained and Controlled Release Drug Delivery Systems J. R. Robinson, ed., Marcel Dekker, Inc., New York, 1978, which is incorporated herein by reference.

Additional active compounds also can be incorporated into the compositions. In certain embodiments, an inhibitory anti-ILT3 antibody of the invention is co-formulated with and/or co-administered with one or more additional therapeutic agents. These agents include, without limitation, antibodies that bind other targets, anti-tumor agents, anti-proliferative agents, chemotherapeutic agents, or peptide analogues that inhibit ILT3. Such combination therapies may require lower dosages of the inhibitory anti-ILT3 antibody as well as the co-administered agents, thus avoiding possible toxicities or complications associated with the various monotherapies.

As noted above, the compositions of the present invention optionally may further comprise a pharmaceutically acceptable antioxidant in addition to a chelating agent. Suitable antioxidants include, but are not limited to, methionine, sodium thiosulfate, catalase, and platinum. For example, the composition may contain methionine in a concentration that ranges from 1 mM to about 100 mM, and in particular, is about 27 mM. For example, an aqueous formulation may be: 10 mg/mL anti-ILT3 antibody, 20 mM Histidine, pH 5.5, 84 mg/mL Trehalose dihydrate, 0.2 mg/mL Polysorbate 80, 0.05 mg/mL disodium EDTA, 0.1 mg/mL L-Methionine.

The compositions of the invention may include a “therapeutically effective amount” or a “prophylactically effective amount” of an antibody or antigen-binding portion of the invention. A “therapeutically effective amount” refers to an amount effective, at dosages and for periods of time necessary, to achieve the desired therapeutic result. A therapeutically effective amount of the antibody or antigen-binding portion may vary according to factors such as the disease state, age, sex, and weight of the individual, and the ability of the antibody or antibody portion to elicit a desired response in the individual. A therapeutically effective amount is also one in which any toxic or detrimental effects of the antibody or antigen-binding portion are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount.

Dosage regimens can be adjusted to provide the optimum desired response (e.g., a therapeutic or prophylactic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the anti-ILT3 antibody or portion thereof and the particular therapeutic or prophylactic effect to be achieved, and (b) the limitations inherent in the art of compounding such an antibody for the treatment of sensitivity in individuals.

An exemplary, non-limiting range for a therapeutically or prophylactically effective amount of an antibody or antibody portion of the invention is 0.025 to 50 mg/kg, more preferably 0.1 to 50 mg/kg, more preferably 0.1-25, 0.1 to 10 or 0.1 to 3 mg/kg. In some embodiments, a formulation contains 5 mg/mL of antibody in a buffer of 20 mM sodium citrate, pH 5.5, 140 mM NaCl, and 0.2 mg/mL polysorbate 80. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition

### Methods of Producing Antibodies and Antibody Producing Cell Lines

### Immunization

In some embodiments, human antibodies are produced by immunizing a non-human, transgenic animal comprising within its genome some or all of human immunoglobulin heavy chain and light chain loci with an ILT3 antigen. In one embodiment, the non-human animal is a XENOMOUSE™ animal. (Abgenix, Inc., Fremont, Calif.).

XENOMOUSE™ mice are engineered mouse strains that comprise large fragments of human immunoglobulin heavy chain and light chain loci and are deficient in mouse antibody production. See, e.g., Green et al., Nature Genetics 7:13-21 (1994) and U.S. Pat. Nos. 5,916,771, 5,939,598, 5,985,615, 5,998,209, 6,075,181, 6,091,001, 6,114,598, 6,130,364, 6,162,963 and 6,150,584. See also WO 91/10741, WO 94/02602, WO 96/34096, WO 96/33735, WO 98/16654, WO 98/24893, WO 98/50433, WO 99/45031, WO 99/53049, WO 00/09560, and WO 00/037504.

In another aspect, the invention provides a method for making anti-ILT3 antibodies from non-human, non-mouse animals by immunizing non-human transgenic animals that comprise human immunoglobulin loci with an ILT3 antigen. One can produce such animals using the methods described in the above-cited documents. The methods disclosed in these documents can be modified as described in U.S. Pat. No. 5,994,619, which is hereby incorporated by reference. U.S. Pat. No. 5,994,619 describes methods for producing novel cultured inner cell mass (CICM) cells and cell lines, derived from pigs and cows, and transgenic CICM cells into which heterologous DNA has been inserted. CICM transgenic cells can be used to produce cloned transgenic embryos, fetuses, and offspring. The '619 patent also describes methods of producing transgenic animals that are capable of transmitting the heterologous DNA to their progeny. In preferred embodiments of the current invention, the non-human animals are mammals, particularly rats, sheep, pigs, goats, cattle or horses.

XENOMOUSE™ mice produce an adult-like human repertoire of fully human antibodies and generate antigen-specific human antibodies. In some embodiments, the XENOMOUSE™ mice contain approximately 80% of the human antibody V gene repertoire through introduction of megabase sized, germline configuration fragments of the human heavy chain loci and kappa light chain loci in yeast artificial chromosome (YAC). In other embodiments, XENOMOUSE™ mice further contain approximately all of the human lambda light chain locus. See Mendez et al., Nature Genetics 15:146-156 (1997), Green and Jakobovits, J. Exp. Med. 188:483-495 (1998), and WO 98/24893, the disclosures of which are hereby incorporated by reference.

In some embodiments, the non-human animal comprising human immunoglobulin genes are animals that have a human immunoglobulin “minilocus”. In the minilocus approach, an exogenous Ig locus is mimicked through the inclusion of individual genes from the Ig locus. Thus, one or more V_{H} genes, one or more D_{H} genes, one or more J_{H} genes, a mu constant domain, and a second constant domain (preferably a gamma constant domain) are formed into a construct for insertion into an animal. This approach is described, inter alia, in U.S. Pat. Nos. 5,545,807, 5,545,806, 5,569,825, 5,625,126, 5,633,425, 5,661,016, 5,770,429, 5,789,650, 5,814,318, 5,591,669, 5,612,205, 5,721,367, 5,789,215, and 5,643,763, hereby incorporated by reference.

In another aspect, the invention provides a method for making humanized ILT3 antibodies. In some embodiments, non-human animals are immunized with an ILT3 antigen as described below under conditions that permit antibody production. Antibody-producing cells are isolated from the animals, fused with myelomas to produce hybridomas, and nucleic acids encoding the heavy and light chains of an anti-ILT3 antibody of interest are isolated. These nucleic acids are subsequently engineered using techniques known to those of skill in the art and as described further below to reduce the amount of non-human sequence, i.e., to humanize the antibody to reduce the immune response in humans

In some embodiments, the ILT3 antigen is isolated and/or purified ILT3. In some embodiments, the ILT3 antigen is a fragment of ILT3. In some embodiments, the ILT3 fragment comprises at least one epitope of ILT3. In other embodiments, the ILT3 antigen is a cell that expresses or overexpresses ILT3 or an immunogenic fragment thereof on its surface. In some embodiments, the ILT3 antigen is a ILT3 fusion protein. In some embodiments, the ILT3 is a synthetic peptide immunogen.

Immunization of animals can be by any method known in the art. See, e.g., Harlow and Lane, Antibodies: A Laboratory Manual, New York: Cold Spring Harbor Press, 1990. Methods for immunizing non-human animals such as mice, rats, sheep, goats, pigs, cattle and horses are well known in the art. See, e.g., Harlow and Lane, supra, and U.S. Pat. No. 5,994,619. In a preferred embodiment, the ILT3 antigen is administered with an adjuvant to stimulate the immune response. Exemplary adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks.

### Production of Antibodies and Antibody-Producing Cell Lines

After immunization of an animal with an ILT3 antigen, antibodies and/or antibody-producing cells can be obtained from the animal. In some embodiments, anti-ILT3 antibody-containing serum is obtained from the animal by bleeding or sacrificing the animal. The serum may be used as it is obtained from the animal, an immunoglobulin fraction may be obtained from the serum, or the anti-ILT3 antibodies may be purified from the serum.

In some embodiments, antibody-producing immortalized cell lines are prepared from cells isolated from the immunized animal. After immunization, the animal is sacrificed and lymph node and/or splenic B cells are immortalized by any means known in the art. Methods of immortalizing cells include, but are not limited to, transfecting them with oncogenes, infecting them with an oncogenic virus and cultivating them under conditions that select for immortalized cells, subjecting them to carcinogenic or mutating compounds, fusing them with an immortalized cell, e.g., a myeloma cell, and inactivating a tumor suppressor gene. See, e.g., Harlow and Lane, supra. If fusion with myeloma cells is used, the myeloma cells preferably do not secrete immunoglobulin polypeptides (a non-secretory cell line). Immortalized cells are screened using ILT3, a portion thereof, or a cell expressing ILT3. In a preferred embodiment, the initial screening is performed using an enzyme-linked immunoassay (ELISA) or a radioimmunoassay. An example of ELISA screening is provided in WO 00/37504, incorporated herein by reference.

Anti-ILT3 antibody-producing cells, e.g., hybridomas, are selected, cloned and further screened for desirable characteristics, including robust growth, high antibody production and desirable antibody characteristics, as discussed further below. Hybridomas can be expanded in vivo in syngeneic animals, in animals that lack an immune system, e.g., nude mice, or in cell culture in vitro. Methods of selecting, cloning and expanding hybridomas are well known to those of ordinary skill in the art.

In a preferred embodiment, the immunized animal is a non-human animal that expresses human immunoglobulin genes and the splenic B cells are fused to a myeloma cell line from the same species as the non-human animal. In one embodiment, the immunized animal is a XENOMOUSE™ mouse and the myeloma cell line is a non-secretory mouse myeloma. In an even more preferred embodiment, the myeloma cell line is P3-X63-Ag8.653 (American Type Culture Collection).

Thus, in one embodiment, the invention provides methods for producing a cell line that produces a human monoclonal antibody or a fragment thereof directed to ILT3 comprising (a) immunizing a non-human transgenic animal described herein with ILT3, a portion of ILT3 or a cell or tissue expressing ILT3; (b) allowing the transgenic animal to mount an immune response to ILT3; (c) isolating antibody-producing cells from transgenic animal; (d) immortalizing the antibody-producing cells; (e) creating individual monoclonal populations of the immortalized antibody-producing cells; and (f) screening the immortalized antibody-producing cells to identify an antibody directed to ILT3.

In another aspect, the invention provides hybridomas that produce a human anti-ILT3 antibody. In a preferred embodiment, the hybridomas are mouse hybridomas, as described above. In other embodiments, the hybridomas are produced in a non-human, non-mouse species such as rats, sheep, pigs, goats, cattle or horses. In another embodiment, the hybridomas are human hybridomas.

In one embodiment of the invention, antibody-producing cells are isolated and expressed in a host cell, for example myeloma cells. In another preferred embodiment, a transgenic animal is immunized with ILT3, primary cells, e.g., spleen or peripheral blood cells, are isolated from an immunized transgenic animal and individual cells producing antibodies specific for the desired antigen are identified. Polyadenylated mRNA from each individual cell is isolated and reverse transcription polymerase chain reaction (RT-PCR) is performed using sense primers that anneal to variable region sequences, e.g., degenerate primers that recognize most or all of the FR1 regions of human heavy and light chain variable region genes and anti-sense primers that anneal to constant or joining region sequences. cDNAs of the heavy and light chain variable domains are then cloned and expressed in any suitable host cell, e.g., a myeloma cell, as chimeric antibodies with respective immunoglobulin constant regions, such as the heavy chain and κ or λ constant domains. See Babcook, J. S. et al., Proc. Natl. Acad. Sci. USA 93:7843-48, 1996, incorporated herein by reference. Anti ILT3 antibodies may then be identified and isolated as described herein.

In another embodiment, phage display techniques can be used to provide libraries containing a repertoire of antibodies with varying affinities for ILT3. For production of such repertoires, it is unnecessary to immortalize the B cells from the immunized animal. Rather, the primary B cells can be used directly as a source of DNA. The mixture of cDNAs obtained from B cell, e.g., derived from spleens, is used to prepare an expression library, for example, a phage display library transfected into E. coli. The resulting cells are tested for immunoreactivity to ILT3. Techniques for the identification of high affinity human antibodies from such libraries are described by Griffiths et al., EMBO J., 13:3245-3260 (1994); Nissim et al., ibid, pp. 692-698 and by Griffiths et al., ibid, 12:725-734, which are incorporated by reference. Ultimately, clones from the library are identified that produce binding affinities of a desired magnitude for the antigen and the DNA encoding the product responsible for such binding is recovered and manipulated for standard recombinant expression. Phage display libraries may also be constructed using previously manipulated nucleotide sequences and screened in a similar fashion. In general, the cDNAs encoding heavy and light chains are independently supplied or linked to form Fv analogs for production in the phage library. The phage library is then screened for the antibodies with the highest affinities for ILT3 and the genetic material recovered from the appropriate clone. Further rounds of screening can increase affinity of the original antibody isolated.

This invention provides a first polypeptide comprising all or a portion of the extracellular domain of ILT3, wherein the polypeptide is water-soluble and does not comprise the Fc portion of an immunoglobulin.

In one embodiment, the polypeptide is isolated. In a further embodiment, the polypeptide comprises the extracellular domain of ILT3. In yet a further embodiment, the polypeptide consists of the extracellular domain of ILT3. Preferably, the ILT3 is human ILT3. In one embodiment, the portion of ILT3 is the IgG1-like domain 1, the IgG1-like domain 2 or the N-terminal 250, 240, 230, 220, 210, 200, 190, 180, 170, 160 or 150 amino acid residues of ILT3. In another embodiment, the portion of the ILT3 is capable of inhibiting T cell proliferation or inducing differentiation of a T cell into a regulatory T cell. Assays to detect T cell proliferation and differentiation into regulatory T cells are well known in the art and include those described below. Such polypeptides are useful for preventing, inhibiting, reducing or suppressing immune responses mediated by the activation of T cells.

Also contemplated are polypeptides of the invention which contain minor variations provided that the variations in the amino acid sequence maintain at least 75%, more preferably at least 80%, 90%, 95%, and most preferably 99% sequence identity and the molecule retains bioactivity (e.g., inhibition of T cell proliferation, differentiation of T cells into regulatory T cells, suppression of immune responses mediated by activated T cells). In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family.

This invention also provides a second polypeptide comprising (i) all or a portion of the extracellular domain of ILT3 operably affixed to (ii) the Fc portion of an immunoglobulin, wherein the Fc portion of the immunoglobulin comprises a function-enhancing mutation, and wherein the polypeptide is water-soluble. The Fc portion may also be substituted with any other peptide that promotes dimerization or oligomerization. For example, the peptide may comprise cysteine residues that form disulfide bonds or other residues that promote covalent or nonconvalent interactions between the peptides such that the peptides mediate dimerization or oligomerization. Suitable peptides include leucine zippers (e.g., those derived from the yeast GCN4 or a modified version thereof. Other exemplary oligomerization domains are described in, e.g., WO 00/69907, WO 99/62953, WO 98/56906, WO 98/18943, and WO 96/37621.

In one embodiment, the polypeptide is isolated. In a further embodiment, the polypeptide comprises the extracellular domain of ILT3. Preferably, the ILT3 is human ILT3. In a further embodiment, the Fc portion of the immunoglobulin is the Fc portion of IgG1. Preferably, the IgG1 is human IgG1. In a further embodiment, the function-enhancing mutation in the Fc portion of the immunoglobulin inhibits the binding of the Fc portion of an immunoglobulin to an Fc receptor. In one example, the function-enhancing mutation in the Fc portion of the immunoglobulin is an Asn->Gln point mutation at amino acid residue 77 of the Fc portion of human IgG1.

This invention further provides a third polypeptide comprising (i) all or a portion of the extracellular domain of ILT3 operably affixed to (ii) a transmembrane domain. In one embodiment the transmembrane domain corresponds to or is derived from the transmembrane domain of human ILT3 (e.g., amino acid residues 259 to 280 of the sequence of GenBank Accession No. U82979). In another embodiment, the transmembrane domain is derived from a protein other than human ILT3 wherein the protein comprises a transmembrane domain. Nucleic acids encoding these polypeptides, expression vectors and host cells comprising the nucleic acids and methods for producing these polypeptides are also provided.

Also contemplated are polypeptides of the invention which contain minor variations provided that the variations in the amino acid sequence maintain at least 75%, more preferably at least 80%, 90%, 95%, and most preferably 99% sequence identity and the molecule retains bioactivity (e.g., inhibition of T cell proliferation, differentiation of T cells into regulatory T cells, suppression of immune responses mediated by activated T cells). In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family.

This invention provides a first isolated nucleic acid which encodes a polypeptide comprising all or a portion of the extracellular domain of ILT3, wherein the polypeptide is water-soluble and does not comprise the Fc portion of an immunoglobulin. This invention includes nucleic acids encoding polypeptides of the invention containing a conservative mutation as described above.

This invention further provides a second isolated nucleic acid which encodes a polypeptide comprising (i) all or a portion of the extracellular domain of ILT3 operably affixed to (ii) the Fc portion of an immunoglobulin, wherein the Fc portion of the immunoglobulin comprises a function-enhancing mutation, and wherein the polypeptide is water-soluble.

In one embodiment of the instant nucleic acids, the nucleic acids are DNA (e.g., cDNA). In a further embodiment, the nucleic acids are RNA.

This invention provides a first expression vector comprising a nucleic acid sequence encoding a polypeptide comprising all or a portion of the extracellular domain of ILT3, wherein the polypeptide is water-soluble and does not comprise the Fc portion of an immunoglobulin.

This invention further provides a second expression vector comprising a nucleic acid sequence encoding a polypeptide comprising (i) all or a portion of the extracellular domain of ILT3 operably affixed to (ii) the Fc portion of an immunoglobulin, wherein the Fc portion of the immunoglobulin comprises a function-enhancing mutation, and wherein the polypeptide is water-soluble.

This invention provides a first host vector system which comprises the first expression vector and a suitable host cell.

This invention further provides a second host vector system which comprises the second expression vector and a suitable host cell.

The polypeptides of the invention, including the antibodies that specifically bind ILT3, may be expressed using any suitable vector. Typically, the vectors are derived from virus, plasmid, prokaryotic or eukaryotic chromosomal elements, or some combination thereof, and may optionally include at least one origin of replication, at least one site for insertion of heterologous nucleic acid, and at least one selectable marker. The invention also contemplates expressing the polypeptides of the invention using artificial chromosomes, e.g., bacterial artificial chromosomes (BACs), yeast artificial chromosomes (YACs), mammalian artificial chromosomes (MACs), and human artificial chromosomes (HACs), e.g., when it is necessary to propagate nucleic acids larger than can readily be accommodated in viral or plasmid vectors.

The vectors will also often include elements that permit in vitro transcription of RNA from the inserted heterologous nucleic acid. Such vectors typically include a phage promoter, such as that from T7, T3, or SP6, flanking the nucleic acid insert. Expression vectors often include a variety of other genetic elements operatively linked to the protein-encoding heterologous nucleic acid insert, typically genetic elements that drive and regulate transcription, such as promoters and enhancer elements, those that facilitate RNA processing, such as transcription termination, splicing signals and/or polyadenylation signals, and those that facilitate translation, such as ribosomal consensus sequences. Other transcription control sequences include, e.g., operators, silencers, and the like. Use of such expression control elements, including those that confer constitutive or inducible expression, and developmental or tissue-regulated expression are well-known in the art.

Expression vectors can be designed to fuse the expressed polypeptide to small protein tags that facilitate purification and/or visualization. Many such tags are known and available. Expression vectors can also be designed to fuse proteins encoded by the heterologous nucleic acid insert to polypeptides larger than purification and/or identification tags. Useful protein fusions include those that permit display of the encoded protein on the surface of a phage or cell, fusions to intrinsically fluorescent proteins, such as luciferase or those that have a green fluorescent protein (GFP)-like chromophore, and fusions for use in two hybrid selection systems.

For long-term, high-yield recombinant production of the proteins, protein fusions, and protein fragments described herein, stable expression is preferred. Stable expression is readily achieved by integration into the host cell genome of vectors (preferably having selectable markers), followed by selection for integrants.

The polypeptides of the invention, including the antibodies that specifically bind ILT3, may be expressed in any appropriate host cell. The host cell can be prokaryotic (bacteria) or eukaryotic (e.g., yeast, insect, plant and animal cells). A host cell strain may be chosen for its ability to carry out desired post-translational modifications of the expressed protein. Such post-translational modifications of the polypeptide include, but are not limited to, acetylation, carboxylation, glycosylation, phosphorylation, hydroxylation, sulfation, lipidation, and acylation.

Exemplary prokaryotic host cells are E. coli, Caulobacter crescentus, Streptomyces species, and Salmonella typhimurium cells. Exemplary yeast host cells are Saccharomyces cerevisiae, Schizosaccharomyces pombe, Pichia pastoris, and Pichia methanolica. Exemplary insect host cells are those from Spodoptera frugiperda (e.g., Sf9 and Sf21 cell lines, and EXPRESSF™ cells (Protein Sciences Corp., Meriden, Conn., USA)), Drosophila S2 cells, and Trichoplusia ni HIGH FIVE® Cells (Invitrogen, Carlsbad, Calif., USA). Exemplary mammalian host cells are COS 1 and COS 7 cells, NS0 cells, Chinese hamster ovary (CHO) cells, NIH 3T3 cells, 293 cells, HEPG2 cells, HeLa cells, L cells, MDCK, HEK293, W138, murine ES cell lines (e.g., from strains 129/SV, C57/BL6, DBA-1, 129/SVJ), K562, Jurkat cells, BW5147 and any other commercially available human cell lines. Other useful mammalian cell lines are well known and readily available from the American Type Culture Collection (ATCC) (Manassas, Va., USA) and the National Institute of General Medical Sciences (NIGMS) Human Genetic Cell Repository at the Coriell Cell Repositories (Camden, N.J., USA).

In one embodiment of the instant host vector systems, the host cell is a eukaryotic, bacterial, insect or yeast cell. In a further embodiment, the host cell is a eukaryotic cell (e.g., a mammalian cell).

This invention provides a method for producing the first polypeptide, comprising (a) culturing the first host vector system under conditions permitting polypeptide synthesis by the host vector system, and (b) recovering the polypeptide so produced.

This invention also provides a method for producing the second polypeptide, comprising (a) culturing the second host vector system under conditions permitting polypeptide synthesis by the host vector system, and (b) recovering the polypeptide so produced.

This invention provides a first composition comprising (a) a pharmaceutically acceptable carrier and (b) the first polypeptide.

This invention further provides a second composition comprising (a) a pharmaceutically acceptable carrier and (b) the second polypeptide.

The polypeptides of the invention have immunosuppressive activity and act on T cells only upon activation. Thus, these polypeptides induce antigen specific tolerance. The polypeptides and compositions of the invention are useful for preventing, inhibiting, suppressing or reducing an immune response mediated by antigen-specific activation of T cells. In one embodiment, the immune response is involved in transplant rejection. In another embodiment, the immune response is associated with an autoimmune disease, hypersensitivity or allergy. In yet another embodiment, the immune response is related to an inflammatory disorder.

This invention provides a method for inhibiting the onset of transplant rejection in a subject who has received, or is about to receive, a transplant, comprising administering to the subject a prophylactically effective amount of the first, second or third polypeptide.

This invention further provides a method for treating transplant rejection in a subject who has received a transplant, comprising administering to the subject a therapeutically effective amount of the first, second or third polypeptide.

In one embodiment of the methods for inhibiting the onset of and treating transplant rejection, the transplant is an organ transplant. In another embodiment, the transplant is a tissue transplant or involves the transplantation of cells. Transplanted organs include, for example, heart, kidney, skin, lung, liver, eye, bone, and bone marrow. Transplanted tissue includes, for example, vascular tissue. Transplanted cells include stem cells, e.g., umbilical cord stem cells or adult stem cells, pancreatic islet cells, epithelial cells, endothelial cells, and liver cells. The transplant may also be a prosthetic device, e.g., stent. The transplant may be xenogeneic or allogeneic. Preferably, the subject is human.

In one embodiment, the polypeptide is administered concurrently with a second immunosuppressive agent, such as cyclosporine, OKT3 Antibody, rapamycin, Campath I, anti-CD69 antibody, thymoglobulin, and anti-thymocytic antibody. The polypeptide may also be administered before or after administration of the second immunosuppressive agent. In another embodiment, the polypeptide is administered at the time of transplantation and twice a week for two weeks as is routine for transplants. In another embodiment, the polypeptide is administered to the subject at the onset of or during rejection.

Symptoms associated with rejection of a transplant are well known in the art and include for kidney, increased blood urea nitrogen (BUN) levels, for pancreas, increased glycemia, for heart, lymphocyte infiltrates, and for liver, increased levels of enzymes such as aspartate aminotransferase (SGOT) and alanine aminotransferase (SGPT).

This invention provides a method for treating a subject afflicted with an autoimmune disorder, comprising administering to the subject a therapeutically effective amount of the first, second or third polypeptide.

The autoimmune disorder treated can be any such disorder, and includes, without limitation, rheumatoid arthritis, Crohn's disease, multiple sclerosis, autoimmune diabetes, systemic lupus erythematosus, lupus vulgaris, thyroiditis, Addison's Disease, hemolytic anemia, antiphospholipid syndrome, dermatitis, allergic encephalomyelitis, glomerulonephritis, Goodpasture's Syndrome, Graves' Disease, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Pulmonary Inflammation, Guillain-Barre Syndrome, and autoimmune inflammatory eye disease. Preferably, in the subject method, the subject is human. In one embodiment, the polypeptide is administered to the subject during a flare-up of an autoimmune attack. The method may further comprise administration of additional immunosuppressive drugs, e.g., cytotoxic agents, cyclosporine, methotrexate, azathioprine, and corticosteroids.

Similarly, allergic reactions and conditions, such as asthma (particularly allergic asthma) or other respiratory problems, may also be treated by a polypeptide of the present invention. These molecules can be used to treat anaphylaxis, hypersensitivity to an antigenic molecule, or blood group incompatibility.

This invention further provides a method for treating a subject afflicted with an inflammatory disorder, comprising administering to the subject a therapeutically effective amount of the first, second or third polypeptide.

The inflammatory disorder treated can be any such disorder, and includes, without limitation, (i) inflammatory diseases such as chronic inflammatory pathologies (including chronic inflammatory pathologies such as, but not limited to, sarcoidosis, chronic inflammatory bowel disease, ulcerative colitis, and Crohn's pathology); (ii) vascular inflammatory pathologies such as, but not limited to, disseminated intravascular coagulation, atherosclerosis, Kawasaki's pathology and vasculitis syndromes (such as, but not limited to, polyarteritis nodosa, Wegener's granulomatosis, Henoch-Schonlein purpura, giant cell arthritis and microscopic vasculitis of the kidneys); (iii) chronic active hepatitis; (iv) Sjogren's syndrome; (v) spondyloarthropathies such as ankylosing spondylitis, psoriatic arthritis and spondylitis, enteropathic arthritis and spondylitis, reactive arthritis and arthritis associated with inflammatory bowel disease; and (vi) uveitis. Preferably, in the subject method, the subject is human. The method can also be combined with administration of additional anti-inflammatory agents. Anti-inflammatory agents include, but are not limited to, any known nonsteroidal anti-inflammatory agent such as, salicylic acid derivatives (aspirin), para-aminophenol derivatives (acetaminophen), indole and indene acetic acids (indomethacin), heteroaryl acetic acids (ketorolac), arylpropionic acids (ibuprofen), anthranilic acids (mefenamic acid), enolic acids (oxicams) and alkanones (nabumetone) and any known steroidal anti-inflammatory agent which include corticosteriods and biologically active synthetic analogs with respect to their relative glucocorticoid (metabolic) and mineralocorticoid (electrolyte-regulating) activities. Additionally, other drugs used in the therapy of inflammation include, but are not limited to, autocoid antagonists such as histamine, bradykinin receptor antagonists, leukotriene and prostaglandin receptor antagonists, and platelet activating factor receptor antagonists.

This invention further provides a method for inducing anergy in a T cell, thereby causing it to differentiate into a regulatory cell, comprising contacting the T cell with the first, second or third polypeptide under conditions permitting priming of the T cell to occur, thereby inducing anergy in the T cell and causing it to differentiate into a regulatory cell. Contacting of the T cell may be performed in vivo, ex vivo or in vitro.

In one embodiment, the T cell is a CD4⁺ T cell, a CD3⁺ cell or a CD8⁺ T cell, and the conditions permitting priming to occur comprise contacting the T cell with an allogeneic stimulator (e.g., an allogeneic antigen presenting cell (APC)) or an autologous APC that has been pulsed with an antigen. Exemplary antigen presenting cells include dendritic cells, monocytes, macrophages, endothelial cells and epithelial cells. In the preferred embodiment, the allogeneic stimulator is an irradiated KG1 cell.

This invention further provides a method for treating a subject afflicted with an autoimmune disorder, comprising contacting, ex vivo, the first, second or third polypeptide with T cells obtained from the subject, wherein the contacting is performed under conditions permitting priming of the cells to occur, and intravenously administering the resulting cells to the subject, so as to treat the subject.

This invention also provides a method for treating transplant rejection in a subject, comprising the steps of contacting, ex vivo, T cells obtained from the subject with a polypeptide of the invention (e.g. the first, second or third polypeptide) under conditions permitting priming of the cells, and administering the resulting cells to the subject.

Methods of treating inflammatory disease or graft versus host disease in a subject by treating T cells obtained from the subject with a polypeptide of the invention ex vivo and then administering the treated T cells to the subject are also contemplated.

In one embodiment, the T cell is a CD4⁺ T cell, a CD3⁺ T cell or a CD8⁺ T cell and the conditions permitting priming to occur comprise contacting the T cell with an allogeneic stimulator (e.g., an allogeneic antigen presenting cell (APC)) or an autologous APC that has been pulsed with an antigen. Exemplary antigen presenting cells include dendritic cells, monocytes, macrophages, endothelial and epithelial cells. In the preferred embodiment, the allogeneic stimulator is an irradiated KG1 cell.

Determining an effective amount of the instant polypeptides for use in the instant invention can be done based on animal data using routine computational methods. In one embodiment, the effective amount, administered intravenously, is between about 0.5 mg/kg and about 50 mg/kg of polypeptide. In another embodiment, the effective amount, administered intravenously, is between about 1 mg/kg and about 20 mg/kg of polypeptide. In the preferred embodiment, the effective amount, administered intravenously, is about 3, 5 or 10 mg/kg of polypeptide. In one embodiment of the instant methods, the polypeptide is administered in a single dose. In another embodiment, the polypeptide is administered in multiple doses.

Determination of a preferred pharmaceutical formulation and a therapeutically efficient dose regiment for a given application is within the skill of the art taking into consideration, for example, the condition and weight of the patient, the extent of desired treatment and the tolerance of the patient for the treatment.

Administration of the polypeptides or compositions of this invention, including isolated and purified forms, may be accomplished using any of the conventionally accepted modes of administration of agents which are used to prevent or treat transplantation rejection or to treat autoimmune or inflammatory disorders. The pharmaceutical composition may comprise any of the antibodies or antigen-binding portions thereof as described above and a pharmaceutically acceptable carrier.

The pharmaceutical compositions of this invention may be in a variety of forms, which may be selected according to the preferred modes of administration. These include, for example, solid, semi-solid and liquid dosage forms such as tablets, pills, powders, liquid solutions or suspensions, suppositories, and injectable and infusible solutions. The preferred form depends on the intended mode of administration and therapeutic application. Modes of administration may include oral, parenteral, subcutaneous, intravenous, intralesional or topical administration.

The compositions of this invention may, for example, be placed into sterile, isotonic formulations with or without cofactors which stimulate uptake or stability. The formulation is preferably liquid, or may be lyophilized powder. For example, the compositions or polypeptides of the invention may be diluted with a formulation buffer comprising 5.0 mg/ml citric acid monohydrate, 2.7 mg/ml trisodium citrate, 41 mg/ml mannitol, 1 mg/ml glycine and 1 mg/ml polysorbate 20. This solution can be lyophilized, stored under refrigeration and reconstituted prior to administration with sterile Water-For-Injection (USP).

The compositions of the present invention can also be formulated so as to provide slow or controlled-release of the active ingredient therein using, e.g., hydropropylmethyl cellulose in varying proportions to provide the desired release profile, other polymer matrices, gels, permeable membranes, osmotic systems, multilayer coatings, microparticles, liposomes and/or microspheres.

In general, a controlled-release preparation is a composition capable of releasing the active ingredient at the required rate to maintain constant pharmacological activity for a desirable period of time. Such dosage forms can provide a supply of a drug to the body during a predetermined period of time and thus maintain drug levels in the therapeutic range for longer periods of time than other non-controlled formulations.

Also provided are methods of delivering membrane-bound polypeptides of the invention using lipid bilayers or by administration of cells manipulated to express the polypeptides of the invention (e.g., cells transfected with a nucleic acid encoding a polypeptide of the invention) (M. Davis et al., JCB 166:579-590 (2004)).

Nucleic acids encoding a polypeptide of the invention may be administered using gene therapy methods. Retrovirus vectors and adeno-associated virus (AAV) vectors are preferred vectors according to the invention for transferring nucleic acids encoding the polypeptides of the invention into cells in vivo, particularly into human cells. These vectors provide efficient delivery of genes into cells, and the transferred nucleic acids are stably integrated into the chromosomal DNA of the host. The development of specialized cell lines (“packaging cells”) that produce replication-defective retroviruses are especially preferred for gene therapy applications (see, e.g., Miller, A. D. Blood 76:271 (1990)). Recombinant retrovirus may be constructed in which part of the retroviral coding sequence (gag, pol, env) has been replaced by nucleic acid encoding one of the subject receptors rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses may be found, e.g., in Current Protocols in Molecular Biology, Ausubel, F. M. et al. (eds.) Greene Publishing Associates, (1989), Sections 9.10-9.14 and other standard laboratory manuals. Representative examples of retroviruses include pLJ, pZIP, pWE and pEM which are well known to those skilled in the art. Representative examples of packaging virus lines for preparing both ecotropic and amphotropic retroviral systems include psi.Crip, psi.Cre, psi 2 and psi.Am. Retroviruses have been widely used to introduce a variety of genes into many different cell types in vitro and/or in vivo. Moreover, it is useful to limit the infection spectrum of retroviruses and retroviral-based vectors by modifying the viral packaging proteins on the surface of the viral particle (see, for example PCT publications WO93/25234 and WO94/06920; Roux et al. PNAS 86:9079-9083 (1989); Julan et al. J. Gen Virol 73:3251-3255 (1992); and Goud et al. Virology 163:251-254 (1983)); Neda et al. J. Biol Chem 266:14143-14146 (1991)).

This invention further provides an article of manufacture comprising (a) a packaging material having therein the first polypeptide, and (b) a label indicating a use for the polypeptide for (i) treating or inhibiting the onset of transplant rejection in a subject, (ii) treating an autoimmune disorder in a subject, or (iii) treating an inflammatory disorder in a subject.

This invention provides an article of manufacture comprising (a) a packaging material having therein the second polypeptide, and (b) a label indicating a use for the polypeptide for (i) treating or inhibiting the onset of transplant rejection in a subject, (ii) treating an autoimmune disorder in a subject, or (iii) treating an inflammatory disorder in a subject.

This invention provides a method of determining tumor metastasis comprising identifying the presence of ILT3+ cells in a sample from the tumor microenvironment or lymph node of a subject, wherein the presence of mILT3 indicates tumor metastasis in the subject. The ILT3+ cells may also be referenced in other manners known to those skilled in the art including but not limited to cells expressing mILT3, and mILT3 cells. The tumor microenvironment may include the stroma.

In one embodiment, the lymph node is the sentinel lymph node. Often, the site of initial cancer spread in various cancers is the local lymph nodes. The sentinel lymph node is often the first lymph node to which cancer is likely to spread from the primary tumor. Accordingly, cancer cells may appear in the sentinel lymph node before spreading to other lymph nodes. The sentinel lymph node sample may be obtained by any means known to those skilled in the art. Such means include a sentinel lymph node biopsy, whereby lymph tissue is removed from the subject.

In one embodiment, the cells are macrophage cells such as tumor associated macrophage cells. In one embodiment, the cells are CD68⁺ cells. The presence of mILT3 may be determined by any means known to those skilled in the art. One means is by immunohistochemical studies to determine ILT3 staining of the cells. Such studies may employ an anti-ILT3 antibody, such as an anti-human ILT3 antibody.

In one embodiment, the subject is afflicted with any cancer known to those skilled in the art, such as the cancers described herein. In one embodiment, the cancer is melanoma. In another embodiment, the cancer is pancreatic carcinoma. In another embodiment, the cancer is colorectal carcinoma.

The invention provides a method of diagnosing cancer in a subject comprising identifying the presence of sILT3 in the subject's serum, wherein the presence of sILT3 diagnoses cancer in the subject. The presence of sILT3 in the serum may be determined by any means known to those skilled in the art. In one embodiment, and ELISA assay, such as a sandwich ELISA, is employed. Such means may employ an anti-ILT3 antibody, such as an anti-human ILT3 antibody. The cancer may be any cancer known to those skilled in the art, including those described herein. In one embodiment, the cancer is melanoma. In one embodiment, the cancer is colorectal carcinoma. In another embodiment, the cancer is pancreatic carcinoma. In another embodiment, the cancer is hematologic malignancy. The hematologic malignancies include but are not limited to cutaneous T cell lymphoma, chronic lymphocytic leukemia, acute lymphocytic leukemia or Hodgkin's Lymphoma.

In one embodiment, the amount of sILT3 in the subject's serum suggesting the presence of cancer in the subject is at least 100 ng/ml. In another embodiment, the amount ranges from 100 to at least 10,000 ng/ml. In another embodiment, the amount is 100-1000 ng/ml. In another embodiment, the amount is 100-500 ng/ml. The amount of sILT3 in the serum may correlate with the stage of subject's cancer. That is, higher amounts of sILT3 suggest more advanced stages of cancer. Accordingly, a higher amount of sILT3 in a subject compared to an amount previously measured in the same subject suggests progression of the cancer to a more advanced stage. In addition, a lower amount of sILT3 in a subject compared to an amount previously measured in the same subject suggests remission of the cancer to a less advanced stage.

The anti-sILT3 antibody may be labeled. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

This invention provides a method of treating a subject afflicted with a disease selected from chronic viral disease and cancer, comprising administering to the subject a composition comprising an RNAi construct targeted to an sILT3 mRNA, in an amount sufficient to attenuate expression of sILT3 by an RNA interference mechanism, thereby treating the subject. The term “RNAi construct” includes small interfering RNAs (siRNAs), hairpin RNAs, and other RNA species which can be cleaved in vivo to form siRNAs. RNAi constructs herein also include expression vectors capable of giving rise to transcripts which form dsRNAs (two strands) or hairpin RNAs (one self complementary strand) in cells, and/or transcripts which can produce siRNAs in vivo. Optionally, the siRNA includes single strands or double strands of siRNA. The siRNAs have significant sequence similarity to a target RNA so that the siRNAs can pair to the target RNA and result in sequence-specific degradation of the target RNA through an RNA interference mechanism. Methods for making and using RNAi constructs are well known in the art and well described in WO01/68836 and WO06/081546.

This invention will be better understood from the Experimental Details which follow. However, one skilled in the art will readily appreciate that the specific methods and results discussed are merely illustrative of the invention as described more fully in the claims which follow thereafter.

### EXPERIMENTAL DETAILS

### First Series of Experiments

The most extensively studied “protein therapeutic drugs” are (1) monoclonal antibodies, (2) cytokine-fusion proteins and (3) chimeric cell adhesion molecules that prevent T cell activation and/or proliferation.

Soluble ILT3 (“sILT3”) (encompassing the first and second instant polypeptides) belongs to the family of chimeric proteins that modulate the immune system. sILT3 is an attractive candidate for therapeutic use based on its potent in vitro immune modulating activities and orthologue (homolog) of ILT3 studies shown in animal models of acute and chronic inflammation and autoimmunity. Soluble ILT3 induces immune tolerance by inhibition of T cell activation that contributes to graft rejection.

Examples of soluble chimeric proteins being investigated in clinical trials are CTLA-4, CD86, PD-1 and PD-L1 fusion proteins. Some have been proven effective against specific autoimmune disease. However, these molecules can behave both in a stimulatory or inhibitory manner depending on their counter-receptors. Furthermore, their expression is ubiquitous such that CTLA-4 and its counter-receptor are expressed on the same T cells. Moreover, the broad expression pattern of their counter-receptors on B cell, DCs, ECs, macrophages, fibroblasts, muscle cells and trophoblast cells render them less-specific for the inhibition of T cell proliferation.

In this respect, ILT3 is unique. Its expression is limited to dendritic (professional) and endothelial (semi-professional) antigen presenting cells, two cell types playing an important role in modulating the immune responses. These cells can be either immunogenic or tolerogenic. However, the expression of ILT3 on these two cell types renders them tolerogenic leading to induction of anergy. ILT3 expression can be induced with inhibitory cytokines (IL10, IFNα) and VitD3 and render DC and EC tolerogenic. However, one can not ignore the possible other, as yet undiscovered effects of cytokine treatments on cells. Thus, directly administering sILT3 such that it directly interact with T cells, rendering them anergic, will be more effective in down-regulating the immune system.

This invention offers several advantages. First, the second polypeptide of this invention has an extended circulating half-life and provides long-term protection, acting as a long-lasting “chimeric” protein drug. The prolonged half-life permits lower dosing, thereby reducing toxicity. Second, soluble polypeptides (i.e. the extracellular domain of ILT3) and longevity-increasing polypeptides (i.e., Fc portion of Ig) useful in this invention can readily be isolated using routine methods.

### Example 1

### Construction of Cells Expressing ILT3 or ILT3Delta

KG1 cells over-expressing human ILT3 (KG1.ILT3 cells) were generated as previously described (CC Chang et al., Nat. Immunol. 3:237-243, 2002). Deletion of the cytoplasmic region of ILT3 was accomplished by PCR amplification using the following primers: sense-5′-CCATGATATCAGGAGACGCCATGATCCCCA-3′ and antisense-5′-ATGTAGCGGCCGCGTTTTCTCCCTGGACGTCA-3′ and a plasmid containing a full-length cDNA of ILT3 (pcDNA4-ILT3) was used as template. PCR conditions were as follows: 5 min 94° C.; 30 cycles (30 sec 94° C., 1 min 68° C., 1 min 72° C.); 7 min 72° C. The PCR product was purified using a PCR purification kit (Qiagen) and subcloned into the EcoRV and NotI sites of the expression vector pcDNA4/TO/myc-His in frame with a c-myc-His epitope (Invitrogen). The resulting ILT3 deletion mutant, ILT3delta (which contains residues 1 (Met) to 328 (Asn) of human ILT3), encodes a protein that contains the putative leader peptide, the extracellular and transmembrane domains and a stretch of 48 amino acids of the cytoplasmic domain of ILT3 followed by a C-terminal myc-His tag. The ILT3delta insert was subcloned into the BglII site of retroviral vector MIG (MSCV-IRES-GFP) and the resulting construct was confirmed by sequencing. The ILT3delta was over-expressed in KG1 cells by retroviral transduction (Change et al., supra). Transfectants were sorted for GFP expression by flow cytometry.

Generation of Soluble ILT3-Fc Chimeric Protein

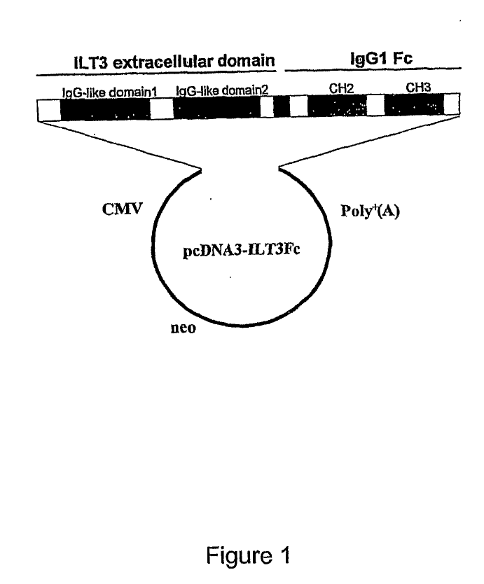

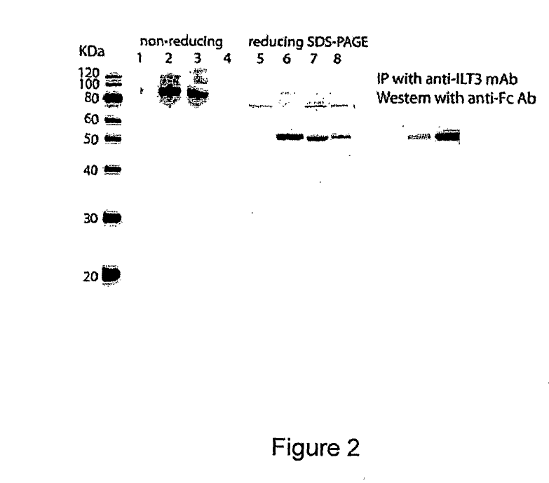

cDNA fragment coding for the extracellular domain of human ILT3 was fused to the Fc portion of the human IgG1 heavy chain. To abolish binding to the Fc receptor, a mutation was introduced in the N-linked glycosylation site, N77 (Asn>Gln) of the Fc domain. Expression vector pcDNA3 (Invitrogen) containing the ILT3-Fc fusion gene (FIG. 1) was transfected into CHO—S cells. Homogenous cell populations were obtained by limiting dilution and clones with high expression of ILT3 (as determined by RT-PCR and Western Blot analysis) were selected. ILT3-Fc fusion proteins were purified from the supernatant of the selected clones using a recombinant protein A FF column and analyzed by Western blotting using an anti-human Fc-specific antibody.

### Example 2

### Membrane ILT3 Induces CD4⁺ T_{H} Cell Anergy and Inhibits the Generation of CD8⁺ Cytotoxic Cells

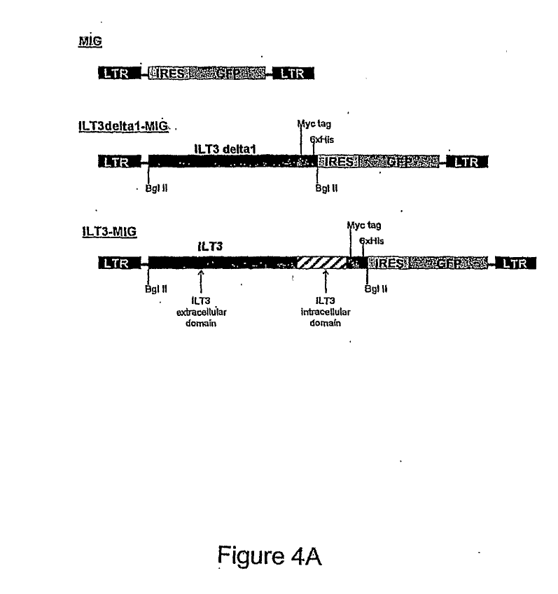

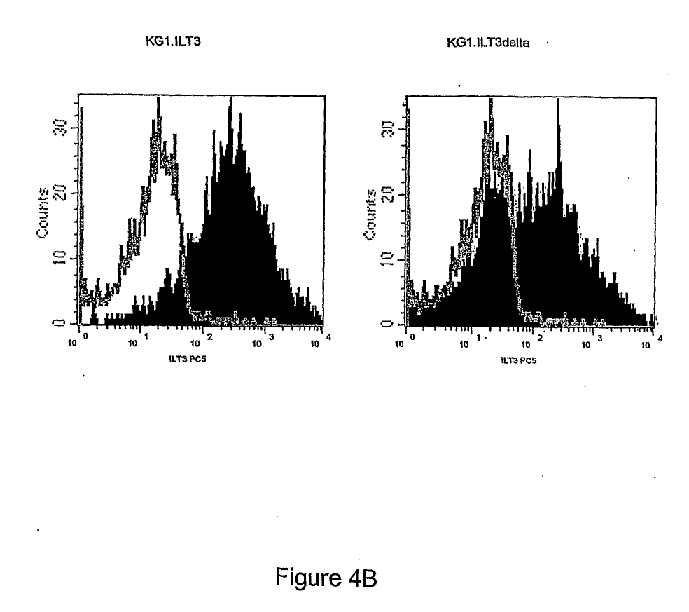

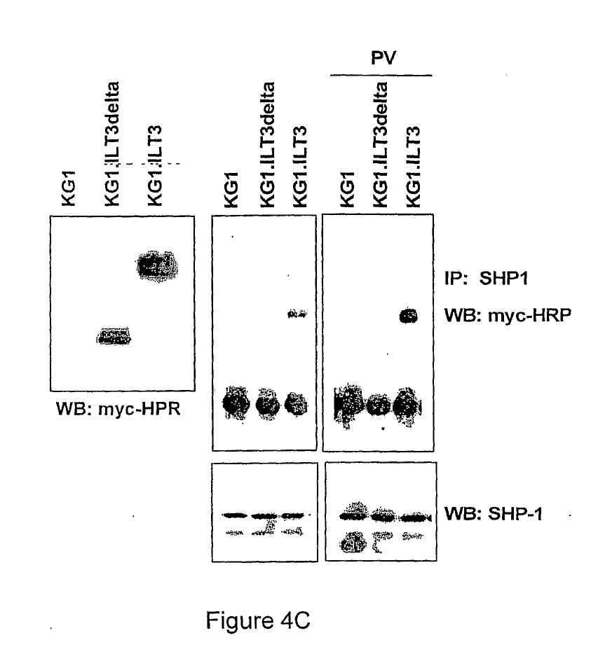

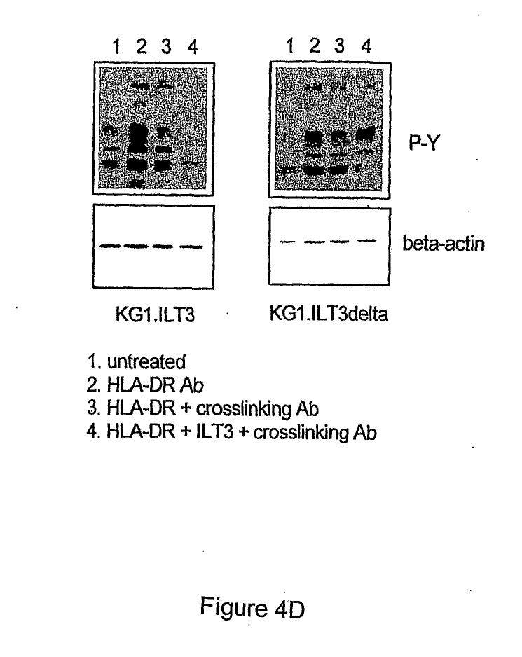

The cytoplasmic region of ILT3 contains ITIM motifs that recruit inhibitory phosphatases, which can negatively regulate cell activation. The extracellular portion contains two Ig domains, one or both of which are likely to contribute to the ILT3 ligand binding sites involved in the interaction of APC with T lymphocytes. The KG1.ILT3 delta cell line was generated by overexpressing the cytoplasmic tail deletion mutant ILT3delta in KG1 cells. This cell line was used to explore the activity of membrane ILT3 (mILT3) (FIG. 4A).

KG1.ILT3 and KG1.ILT3delta expressed similar amounts of ILT3 protein on the cell surface as shown by flow cytometry analysis using mAb to ILT3 (FIG. 4B). Western blot analysis using anti-myc antibody, which binds to the C-terminal myc tag of the recombinant proteins, demonstrated that the molecular weight of the ILT3delta protein was 38 kDa while that of full length ILT3 was 50 kDa (FIG. 4C).

ILT3 associates with SHP-1 phosphatase and this association is increased by receptor crosslinking with specific antibodies or with pervanadate treatment. SHP-1 recruitment upon phosphorylation of the cytoplasmic ITIMs has been shown to mediate the negative signaling of ILT2, a closely related member of the same family of Ig-like inhibitory receptors as ILT3. Immunoprecipitation experiments using the anti-SHP-1 antibodies and western blot analysis using an anti-myc antibody showed constitutive interaction of the full-length ILT3 molecule and SHP-1. As expected, no interaction of SHP-1 with ILT3delta was observed (FIG. 4C).

Antibodies against HLA-DR can trigger both Ca2⁺ mobilization and specific protein phosphorylation. Co-crosslinking of the anti-HLA-DR antibody with anti-ILT3 antibody results in substantial inhibition or more rapid extinction of the activation signal (Cella et al., J. Exp. Med., 185:1743-1751, 1997). To establish whether lack of SHP-1 recruitment by ILT3delta was accompanied by a lack of inhibition of protein tyrosine phosphorylation, KG1.ILT3 and KG1.ILT3delta cells were ligated with anti-HLA-DR mAb or with anti-HLA-DR mAb and anti-ILT3 mAb in the presence of a crosslinking antibody. Total cell extracts were analyzed by western blot with anti-phoshotyrosine mAb and reprobed with anti-α-actin mAb for control of equal loading. The results showed that crosslinking HLA-DR and ILT3 on KG1.ILT3 cells inhibits tyrosine phosphorylation, however, it has little or no effect on tyrosine phosphorylation in KG1.ILT3delta mutants (FIG. 4D).

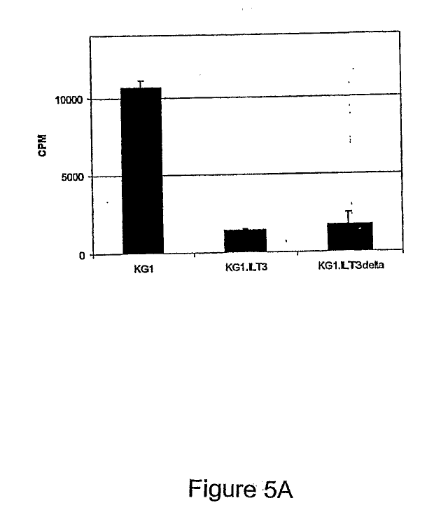

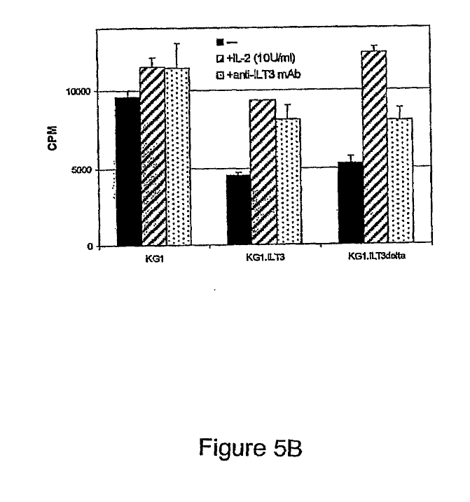

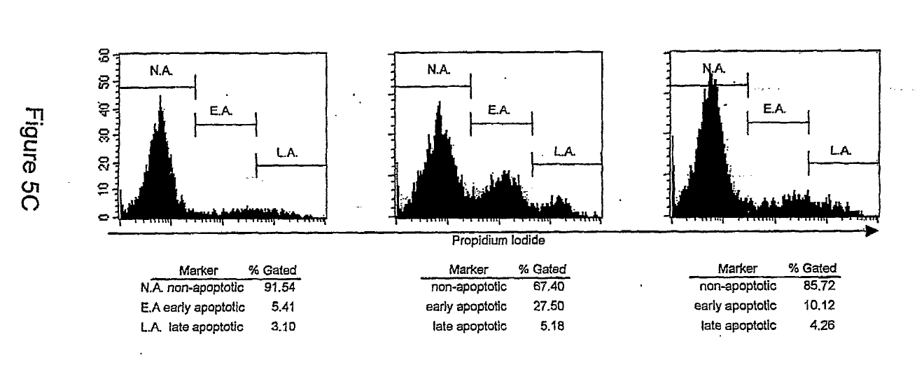

Comparison of the capacity of KG1, KG1.ILT3 and KG1.ILT3delta to elicit T cell proliferation in primary and secondary MLC showed that KG1.ILT3 and KG1.ILT3delta elicited much less proliferation of unprimed (FIG. 5A) or KG1-primed T cells (FIG. 5B) than KG1 cells.

For the proliferation assays, responding T cells (5×10⁴/well) were tested for reactivity to irradiated KG1, KG1.ILT3, KG1.ILT3delta, or allogeneic CD2-depleted APC (2.5×10⁴/well). After 5 days (for naive T cells) or 2 days (for primed T cells) of incubation, the cultures were pulsed with [³H]-thymidine and harvested 18 hours later. [³H]-thymidine incorporation was determined by scintillation spectrometry in an LKB 1250 Betaplate counter. Mean counts per minute (c.p.m.) of triplicate cultures and the standard deviation (sd) to the mean were calculated.

Addition of anti-ILT3 monoclonal antibody (5 μg/ml) or IL-2 (10 U/ml) to the blastogenesis assays restored T cell proliferation in response to KG1.ILT3 and KG1.ILT3delta (FIG. 5B). These experiments indicate that mILT3 protein is sufficient for inducing an inhibitory signal in activated T cells and that deletion of the cytoplasmic region of ILT3 does not abrogate its T cell anergizing activity.

To study the effect of ILT3 on the generation of cytotoxic T cells, CD3⁺CD25-T cells were primed with KG1, KG1.ILT3 or KG1.ILT3delta. After 7 days, CD8⁺ T cells were isolated from each of the cell cultures and tested for their ability to kill KG1 cells. T cells primed with KG1.ILT3 or KG1.ILT3delta showed significantly less cytotoxic activity (6%) than T cells primed with KG1 (24%) as determined by Annexin V/Propidium Iodine staining (FIG. 5C) and produced less IFN-gamma. Thus, T cell interaction with mILT3 inhibits the differentiation of cytotoxic effector cells.

sILT3 Induces CD4⁺ T_{H} Cell Anergy and Inhibits the Generation of CD8⁺ Cytotoxic Cells

sILT3 expression was assayed by Western blotting. Briefly, cell extracts at equal concentration were immunoprecipitated from cleared extract using mouse anti-ILT3 mAb (ZM 3.8) and subjected to SDS-PAGE. Proteins were then electrotransferred onto polyvinylidene difluoride (PVDF) membrane, and were incubated with anti-human Fc polyclonal antibody. Immunoblots were developed by ECL and acquired by the phosphor/fluorescence imager. Apparent molecular weight of soluble ILT3 is about 90 and 50 kDA in non-reducing and reducing conditions, respectively.

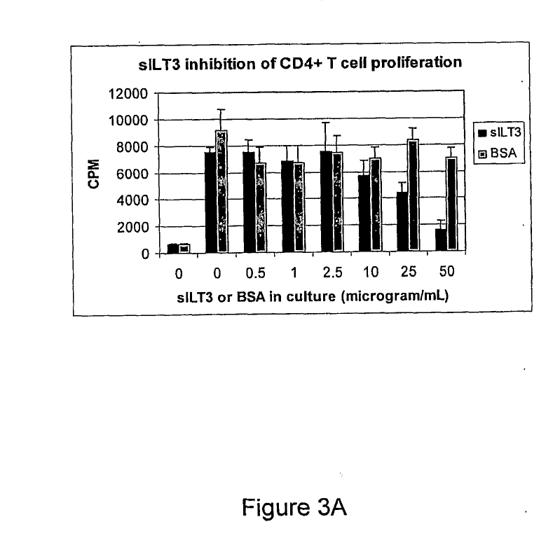

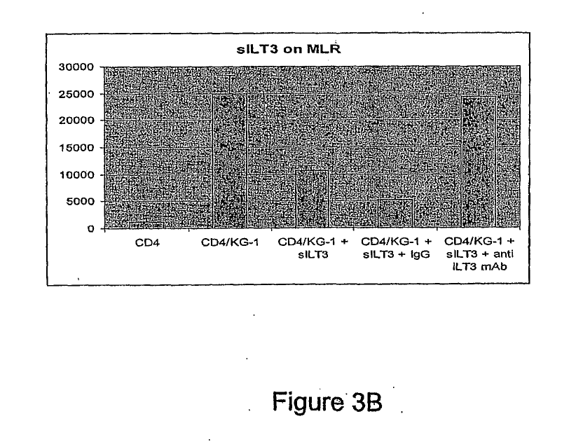

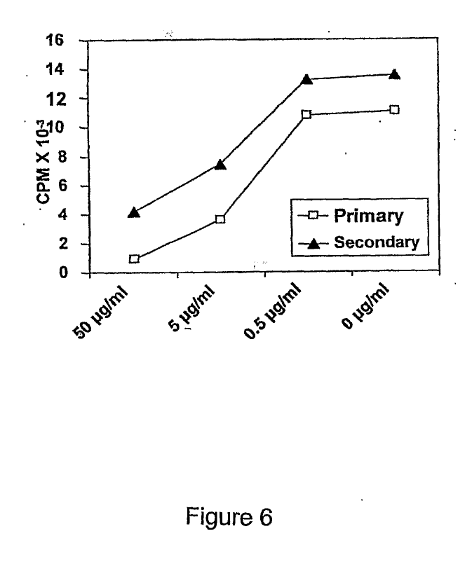

Responding CD3+CD25- or CD4⁺CD25⁻ cells (1×10⁵ cells/well) were stimulated with irradiated KG-1 cells (30 min) (0.5×10⁵ cells/well) in the presence or absence of sILT3-Fc fusion protein in 96-well, round bottom microtiter plates. At 5 days later, cell proliferation was determined by pulsing with [³H]thymidine (0.5 μCi/well) overnight and radioactivity was counted on a beta reader. Addition of 50 μg/ml sILT3 inhibited cell proliferation by >90% (FIGS. 3 and 6). Similarly, when 50 μg/ml sILT3 was added to secondary MLC at the time of restimulation there was >70% inhibition of the secondary response (FIG. 6).

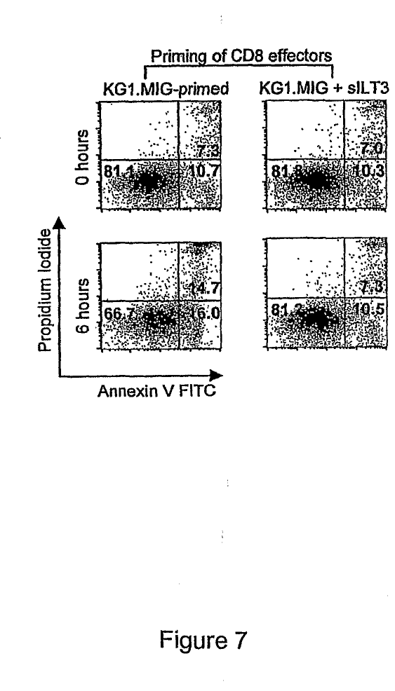

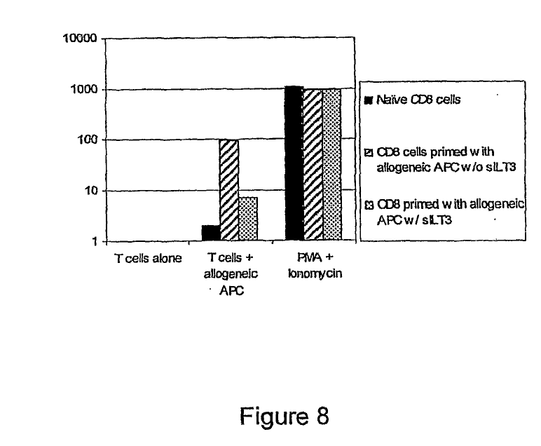

Analysis of the capacity of sILT3 to inhibit generation of cytotoxic T cells showed that CD8⁺ T cells primed in 7-day cultures with KG1 cells, in the presence of sILT3 (50 μg/ml) were devoid of killing capacity (FIG. 7). Furthermore, CD8⁺ T cells from these cultures did not produce IFN-gamma as demonstrated in ELISPOT assays (FIG. 8). Taken together these data indicate that sILT3, similar to mILT3, induces T_{H} anergy and blocks the generation of cytotoxic T cells.

### Example 3

### mILT3 and sILT3 Induce the Generation of Regulatory/Suppressor T Cells

The finding that sILT3 inhibits T cell proliferation in response to allogeneic stimulating cells suggested the possibility that this protein induces anergy in primed T cells triggering their differentiation into regulatory cells.

To explore this hypothesis, unprimed CD4⁺ or CD8⁺ T cells were incubated with allogeneic stimulators (irradiated KG1 cells) in the presence or absence of sILT3 (50 μg/ml). After 7 days, T cells were harvested from the cultures and tested for: (a) expression of FOXP3 (a T suppressor/regulatory cell marker) and (b) capacity to inhibit MLC reactions.

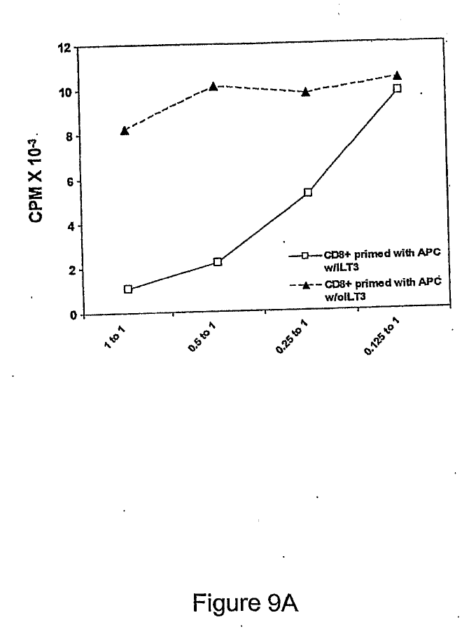

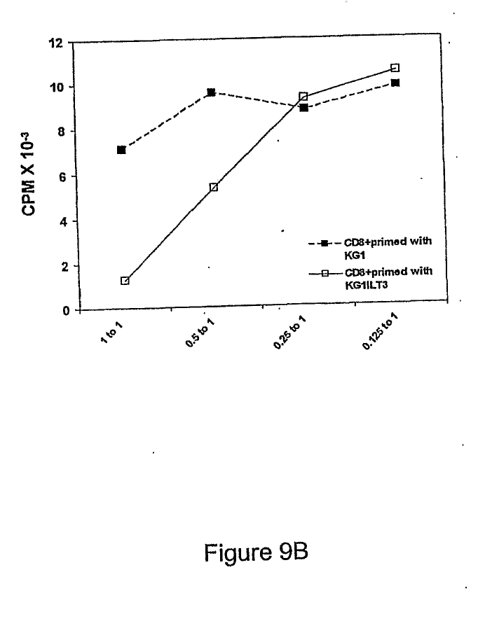

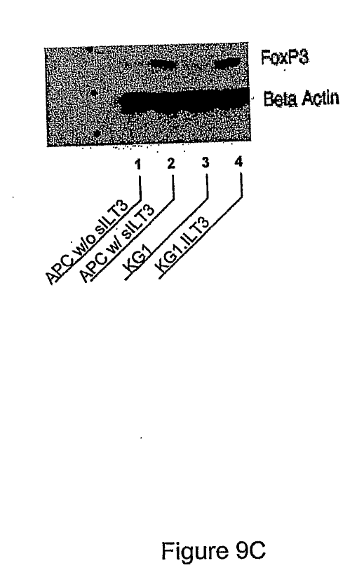

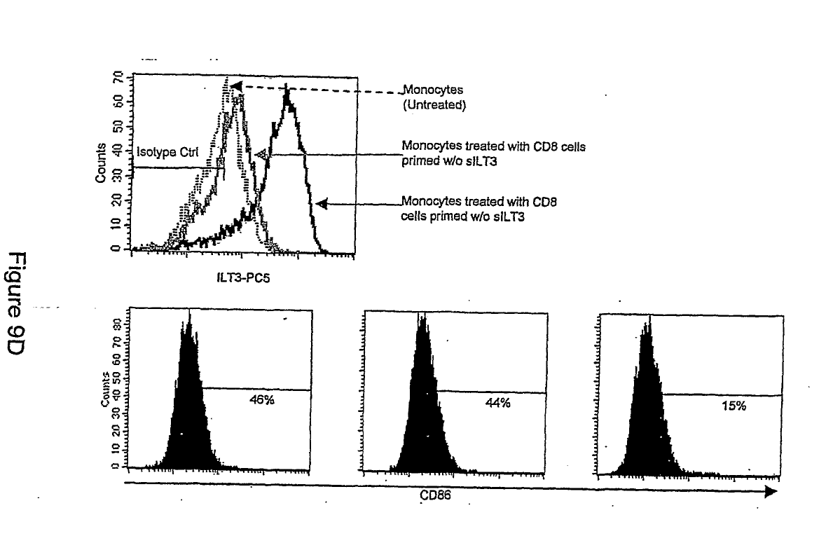

CD4⁺ and CD8⁺ T cells primed in the presence of sILT3 for 7 days expressed FOXP3 and suppressed KG1-triggered proliferation of naïve CD4⁺ T cells in MLC. Control T cells primed for 7 days in cultures with sILT3 or with sILT3 but without allogeneic stimulating cells did not acquire regulatory function. CD8⁺ T cells primed in the presence of sILT3 induced dose-dependent inhibition of T cell proliferation from 50% at a 0.25:1 ratio of primed CD8⁺ T_{S} to responding CD4⁺ T_{H} cell ratio to 90% at a 1:1 ratio (FIG. 9A). CD8⁺ T cells primed in cultures without sILT3 induced 20% inhibition at the highest concentration and virtually no inhibition at lower doses.

The capacity of mILT3 to induce the generation of CD8⁺ T suppressor cells was also tested. CD3+CD25⁻ T cells were primed for 7 days either with KG1 or KG1-ILT3delta cells and then CD8⁺ T cells were isolated and tested for their capacity to inhibit the response of unprimed, autologous CD4⁺ T cells to KG1. CD8⁺ T cells primed to KG1-ILT3 induced dose-dependent inhibition of T cell response to KG1 while CD8⁺ T cells primed to KG1 showed inhibitory activity (35%) only at a 1:1 ratio (FIG. 9B).

Therefore sILT3 as well as mILT3 induces the differentiation of CD8⁺ T suppressor cells in primary MLC. Since FOXP3 is a characteristic marker for CD4⁺ and CD8⁺ regulatory T cells, its expression in CD8⁺ T cells primed for 7 days to allogeneic APC in the presence or absence of sILT3 (50 μg/ml) was determined. CD8⁺ T cells from cultures stimulated either with KG1 or with KG1.ILT3delta were also tested for FOXP3 expression. Western Blot analysis using mAb to FOXP3 (FIG. 9C) showed that both sILT3 and mILT3 induced CD8⁺ T cells with suppressor activity and high expression of FOXP3. Taken together these data indicate that both sILT3 and mILT3 induce the differentiation of CD8⁺ T_{S} with potent inhibitory activity.

### Example 4

### sILT3 Induces the Generation of CD8⁺ T Suppressor Cells by Interaction with CD4⁺ T Cells

CD8⁺ T suppressor cells generated by multiple in vitro stimulation with allogeneic APC were previously shown to act directly on APC, inducing the down regulation of costimulatory molecules and the upregulation of ILT3 and ILT4. To determine whether T suppressor cells generated by allostimulation in the presence of sILT3 have a similar effect on APC, we tested CD8⁺ T cells primed under such conditions for their ability to modulate CD86 and ILT3 expression on DC from the donor used for priming and on control DC from an individual sharing no HLA class I antigens with the original stimulator.

CD8⁺ T cells isolated from the culture containing sILT3 were able to dramatically upregulate the expression of ILT3 on DC from the specific stimulator but not on control APC. This alloantigen-specific upregulation of the inhibitory receptor ILT3 occurred in conjunction with the downregulation of CD86 (FIG. 9D).

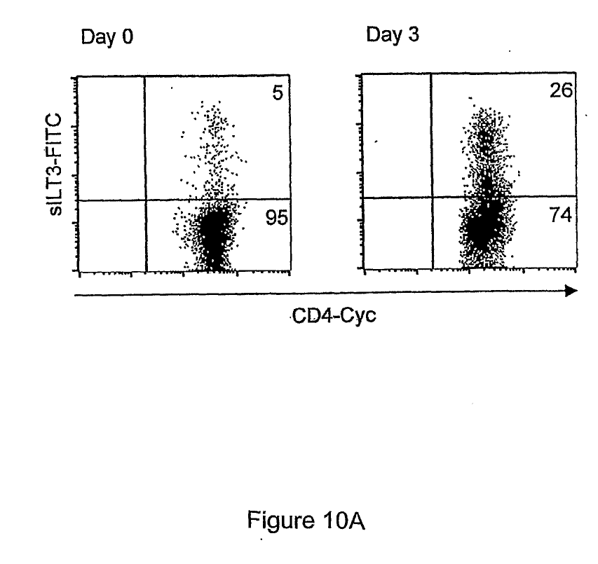

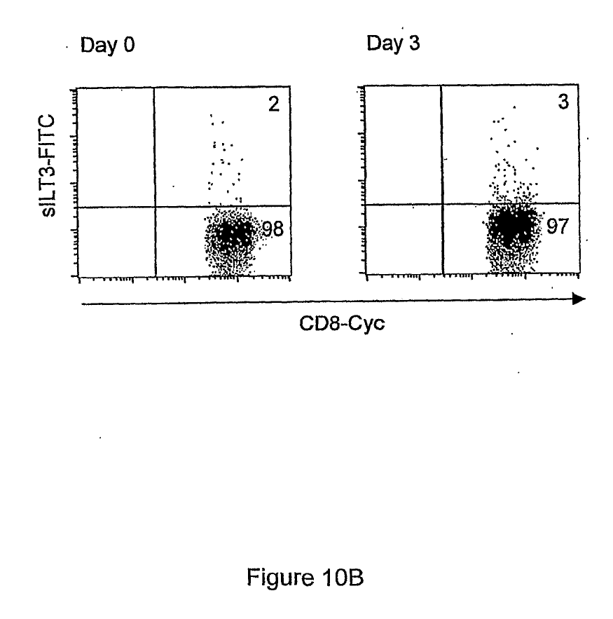

To determine if the generation of CD8⁺ T suppressor cells is due to direct interaction of ILT3 with CD8⁺ T cells or if it results indirectly from interaction between ILT3 and CD4⁺ T cells, FITC-labeled sILT3 protein was used to stain T cells in primary MLC. CD4⁺ T cells, but not CD8⁺ T cells were stained by sILT3-FITC indicating that direct interaction of ILT3 and CD8⁺ cells is not involved in the generation of CD8⁺ T suppressor cells (FIG. 10).

The discovery that sILT3 has potent immunosuppressive activity and that it acts on T cells only upon activation has important clinical implication indicates that sILT3 is useful for inducing antigen specific tolerance.

sILT3 signaling in T lymphocytes via its ligand may interfere with the generation of effective immunity, promoting the generation of T cells with suppressive function. Because sILT3 has no effect on resting, non-stimulated T cells it is likely to inhibit allograft rejection mediated by T cells activated via direct or indirect pathways. Administration of sILT3 can also attenuate the proliferation of T cells involved in aggressive autoimmune responses triggered by activated DC which cross present immunogenic self-peptides. Attenuation of activation efficiency may result in stalled immune responses which perpetuate quiescence.

These findings have important clinical implications because they open two new avenues for antigen-specific suppression of the immune response in transplantation and autoimmune diseases.

The first avenue resides in treating transplant recipients at the time of or after transplantation or at the onset of an acute rejection episode with sILT3. This molecule is expected to bind only to T cells that have been activated by the transplant's alloantigens and not to unprimed T cells, thus suppressing the immune response in an antigen-specific manner. Similarly, sILT3 administration during the flare-up of an autoimmune attack, e.g., in rheumatoid arthritis, Crohn disease, multiple sclerosis, or onset of type I JDM, may prevent the evolution, e.g., progression of the disease.

The second avenue is to use cell therapy, by leukophoresing the patient and exposing the harvested cells to sILT3 for 18 h. In vitro activated T cells, but not unprimed T cells, are expected to be converted into regulatory cells which, when reinfused into the patient, should block the progression of the immune response.

### Example 5

### Membrane and Soluble sILT3 Induces Tolerance to Allogeneic Tumors in Humanized SCID Mice

In the previous studies, it was shown that membrane ILT3 (mILT3) and soluble ILT3 (sILT3) inhibit T cell activation and effector function while eliciting the differentiation of antigen-specific T suppressor cells (Ts) in vitro. To determine the effect of mILT3 and sILT3 on the immune response in vivo, a humanized SCID mouse model was used.

### Methods

SCID mice from Taconic (NY), 8 to 10 wks old, were injected i.p. with 300×10⁶ human PBMC and concomitantly grafted with either KG1 or KG1.ILT3 by S.C. injection of 2×10⁶ cells. Tumor growth was inspected daily and measured at 2 day intervals. One group of mice grafted with KG1 cells (and injected i.p. with human PMBC) received daily doses of 250 μg of ILT3-Fc administered i.p. from day 0 (day of transplantation) to day 11.

### Results and Discussion

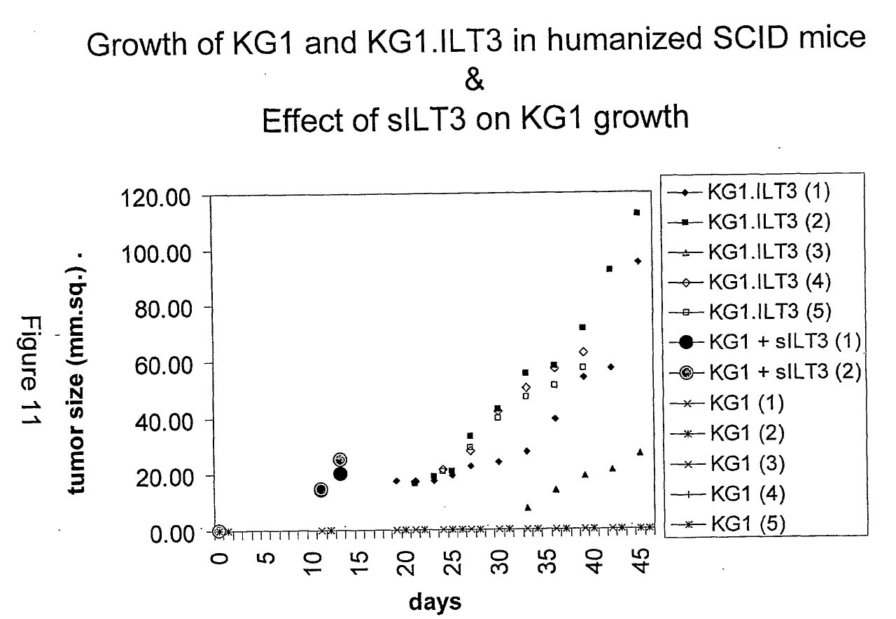

Humanized SCID mice transplanted with KG1 showed no tumor growth over 60 days of observation (0/5). See FIG. 11. Humanized SCID mice transplanted with KG1.ILT3 showed tumor growth 20 to 30 days after transplantation (5/5). Tumor continued to grow to lethal sizes. See FIG. 11.

Human SCID mice transplanted with KG1 and treated for 11 days with sILT3 showed tumor growth 11 days post-transplantation. See FIG. 11. These data demonstrate that sILT3 is a potent immunosuppressive agent. The implication of these results is that sILT3 is a component of tolerance to a tumor, allowing unfettered tumor growth, and that clearance of sILT3 from the subject would reduce tolerance to the tumor.

### Example 6

### Soluble sILT3 is Present in the Circulation of Some HIV Infected Individuals

In previous studies, it was first demonstrated that HIV infected individuals display high expression of inhibitory molecules on their circulating monocytes, accounting for their poor antigen presenting capacity. The possibility was explored that these inhibitory molecules are also present in the circulation.

### Methods

To determine whether ILT3 is present in patients' sera, both sandwich and direct ELISA were used. For “sandwich ELISA” the capture antibody (gift from Dr. Marco Colonna) was coated on 96-well plates overnight at 4° C. Plates were blocked with PBS containing bovine sera albumin and then washed with PBS/Tween. Patients' sera were plated at various dilutions. After 1 hour of incubation plates were washed again with PBS/Tween and the biotinylated detection antibody (R&D Systems) was added. After 1 hour of incubation, plates were washed and developed using ABTS substrate.

For direct ELISA, serial dilution of patients' sera were coated directly on the plates and ILT3 was detected as above.

### Results

ILT3 was not detected in the serum of healthy volunteers (0/6). However, 28% of HIV infected individuals (2/7) showed ILT3 in the serum. This finding suggests that that serum ILT3 may contribute to inhibition of T-cell mediated immune responses in patients with HIV.

### REFERENCES FOR FIRST SERIES OF EXPERIMENTS

1. “Leukocyte Immunoglobulin-like Receptor, Subfamily B, Member 4; LILRB4,” OMIM 604821 (2000).
2. M. Colonna, et al., U.S. Patent Publication No. 20030165875, published Sep. 4, 2003.

3. B. G. Beinhauer, et al., “Interleukin 10 regulates cell surface and soluble LIR-2 (CD85d) expression on dendritic cells resulting in T cell hyporesponsiveness in vitro”, Eur. J. Immunol. 34:74-80 (2004).

### Second Series of Experiments

The development of new strategies to promote immune responses in certain viral diseases and malignancies or alternatively, to suppress their activation in autoimmune diseases and transplantation is critical to overcome the limited efficacy of conventional therapies. Research in this area has been fueled by the discovery that regulatory T cells and tolerogenic Antigen Presenting Cells (APC) modulate the immune response to self and non-self antigens. The concept has emerged that bi-directional interactions between APC and antigen experienced T cells can initiate either a tolerogenic or an immunogenic pathway.(1-3)

There is increasing evidence that the immune response can be inhibited by various CD4⁺ and CD8⁺ regulatory T (T_{R}) cells, which participate in innate and adaptive immunity.(3,4)

Naturally arising CD4⁺ and CD8⁺ T_{R} develop during the normal process of T cell maturation in the thymus, play an essential role in preventing autoimmune diseases, and are characterized by the constitutive expression of CD25 (the IL-2R α-chain) and fork-head-winged helix transcription factor FOXP3. After T cell receptor (TCR) triggering, natural T_{R} cells inhibit immune responses in vivo and in vitro in an antigen-nonspecific, APC-independent and MHC-nonrestricted manner. Natural T_{R}, are anergic, do not produce cytokines and suppress effector T cells by cell-to-cell contact. (5-10)

Adaptive CD4⁺ and CD8⁺ T_{R} cells are antigen induced, develop in the periphery, and exert their function either by secreting inhibitory cytokines (such as IL-10 and TGF-) or tolerizing directly the APC with which they interact.(9-11) In humans, tolerogenic APC express high levels of inhibitory receptors such as immunoglobulin like transcript (ILT)₃ and ILT4, induce T cell anergy and elicit the differentiation of antigen-specific CD4 and CD8 regulatory T cells.(12,13) For the sake of consistency, CD8⁺ T regulatory cells will be referred to as T suppressor cells (Ts). (3,11-13) The expression of ILT3 and ILT4 was shown to be confined to dendritic cells, monocyte and macrophages. (14-16)

In previous studies it was shown that chronic in vitro stimulation of human T cells with peptide-pulsed autologous APC or with allogeneic APC results in the generation of MHC class I restricted CD8⁺ Ts which inhibit the activation and effector function of T helper (Th) and cytotoxic (Tc) cells with cognate specificity.(3, 11-13) Alloantigen specific CD8⁺ Ts induce the upregulation of ILT3 and ILT4 on monocytes and dendritic cells (DC) rendering them tolerogenic.(12) Tolerogenic ILT3^{high} ILT4^{high} DC induce anergy in alloreactive CD4⁺CD45RO⁺CD25⁺ T cells converting these cells into regulatory T cells (Treg) which perpetuate the suppressor cell cascade by tolerizing other APC. (13)

It was recently demonstrated that the extracellular region of ILT3 is endowed with immunomodulatory properties. Both membrane bound (m) and soluble (s) ILT3 inhibited T cell proliferation in MLC, anergizing CD4⁺ Th cells, suppressing the differentiation of IFN-γ-producing CD8⁺ Tc and inducing the differentiation of alloantigen specific CD8⁺ Ts in primary 7-day MLC. (17)

To substantiate in vivo the immunosuppressive property displayed by sILT3 in vitro, SCID mice were “humanized” (hu-SCID) by injection of human peripheral blood mononuclear cells (PBMC), and then these hu-SCID mice were transplanted with human tumors. While tumors were rejected acutely by hu-SCID mice, they grew explosively in animals treated with ILT3-Fc. It is report here that both membrane and soluble ILT3 induce tolerance to allogeneic tumor transplants in hu-SCID mice, eliciting the differentiation of CD8⁺ human Ts.

In view of recent studies unraveling the role of Tr/Ts and tolerogenic DC in tumor growth and metastasis, the possibility that membrane and soluble ILT3 participate in the induction of T cell anergy and differentiation of regulatory T cells in patients with cancer was explored. (9-10, 18-21)

It is now reported that both s and mILT3 induce tolerance to allogeneic tumor transplants in “humanized” SCID mice (hu-SCID). Furthermore, sera from patients with melanoma, carcinoma of the pancreas, colon and rectum contain sILT3 which promotes the differentiation of Ts and inhibit T cell reactivity in vitro. Understanding the role of ILT3 in progression of malignant diseases opens the way to new therapeutic approaches.

### Results

### ILT3 Induces Tolerance to Allogeneic Tumors

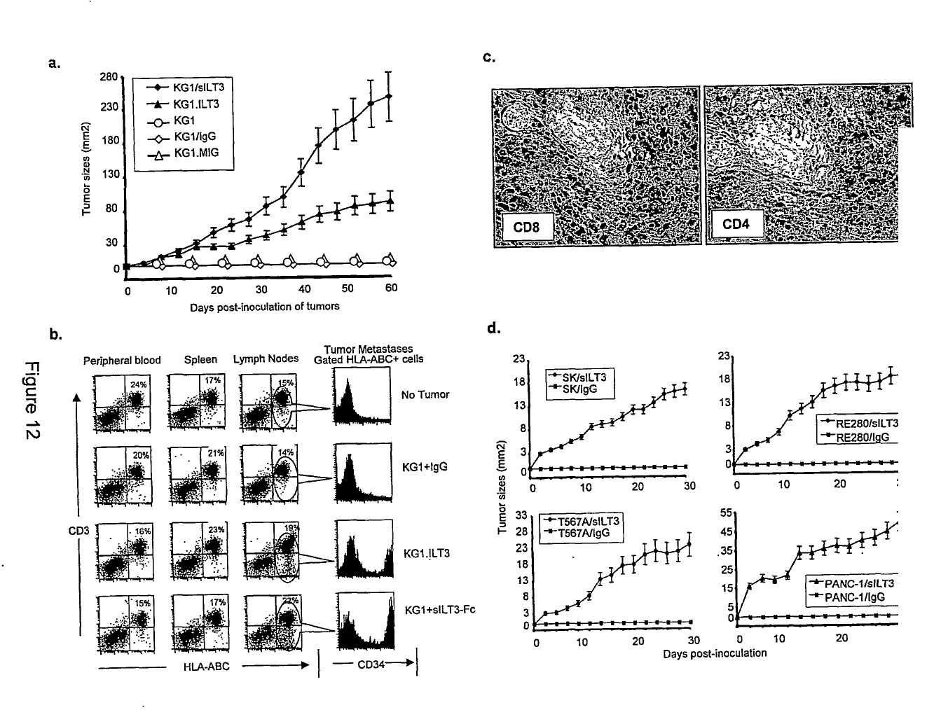

To explore the immunomodulatory effect of s and mILT3, hu-SCID mice were transplanted with the myelomonocytic cell line KG1 (“wild-type” KG1), KG1.MIG (transfected with empty vector), or KG1.ILT3 (transfected with full-length mILT3).(12) In non-humanized SCID mice all tumors grew at roughly the same rate (data not shown). Groups consisting of 10 hu-SCID mice each received: 1) “wild-type” KG1 2) KG1.MIG 3) KG1.ILT3 4) “wild-type” KG1 plus human IgG throughout the first 10 days post-transplantation 5) wild-type KG1 plus rILT3-Fc for days post-transplantation. Subcutaneous transplants of wild-type KG1 or KG1.MIG were rejected by hu-SCID hosts, generating no tumor (group 1 and 2). In contrast, when KG1-ILT3 cells were transplanted s.c. in hu-SCID mice (group 3), they generated tumors that grew to a large size (≧50 mm² cross-sectional area) within two months. Furthermore, hu-SCID mice transplanted with “wild-type” KG1 cells and treated with rILT3-Fc protein (group 5) developed tumors that grew aggressively reaching a cross-sectional area of 240 mm² within 60 days. Control hu-SCID mice transplanted with wild-type KG1 and treated with human IgG (group 4) developed no tumors (FIG. 12A). The data suggested that rILT3-Fc as well as membrane ILT3 inhibited the capacity of human T cells to reject the allogeneic KG1 tumor transplant.

To exclude the possibility that the fate of the transplanted tumor cells was determined by the number of human T cells that reached the periphery, rather than by their exposure to ILT3, T cell engraftment in mice from each experimental group was monitored. The percentage of human CD3⁺ T cells varied from mouse to mouse within each group, but there were no obvious differences between hu-SCID mice without tumor transplants, with rejected or tolerated tumor grafts. Lymph nodes draining the growing KG1 or KG1.ILT3 tumors contained, in addition to human T cells, a population of CD3⁻CD34⁺ HLA class I⁺ cells which were most likely metastatic tumor cells. The spleens of these tumor bearing mice, however, showed no metastatic KG1 cells (FIG. 12B).

Immunohistochemical examination of KG1 tumors growing in hu-SCID hosts showed focal infiltrates of CD8⁺ and diffuse but sparse CD4⁺ T cell infiltrates. There was no tumor cell necrosis or changes suggestive of rejection in the proximity of these infiltrates (FIG. 12C) suggesting that T cells infiltrating the tumor were rendered anergic or converted into Tr/Ts upon exposure to ILT3. Soluble ILT3 had a much stronger tolerogenic effect than mILT3 (expressed by KG1.ILT3) probably because of its systemic, early and continuous access to human T cells migrating toward the tumor.

The tolerogenic effect of ILT3-Fc on hu-SCID mice transplanted with human tumors was further proven in experiments for which we used melanoma (SK-ME-28) and pancreatic carcinoma cell lines such as PANC-1, RE280 and T567A. Untreated hu-SCID mice rejected the tumors, while ILT3-Fc treated mice showed rapid tumor growth demonstrating unambiguously that soluble ILT3 inhibits the capacity of human T cells to reject allogeneic tumor transplants (FIG. 12D).

### Soluble and Membrane ILT3 Induce CD8⁺ T_{S}

In previous studies it was shown that CD8⁺ T cells, allostimulated in the presence of m or sILT3, not only lose cytotoxic activity but also acquire suppressor function.(17) To determine whether this phenomenon also occurs in vivo, the suppressor activity of human T cells from hu-SCID mice sacrificed 8 weeks following tumor transplantation was tested.

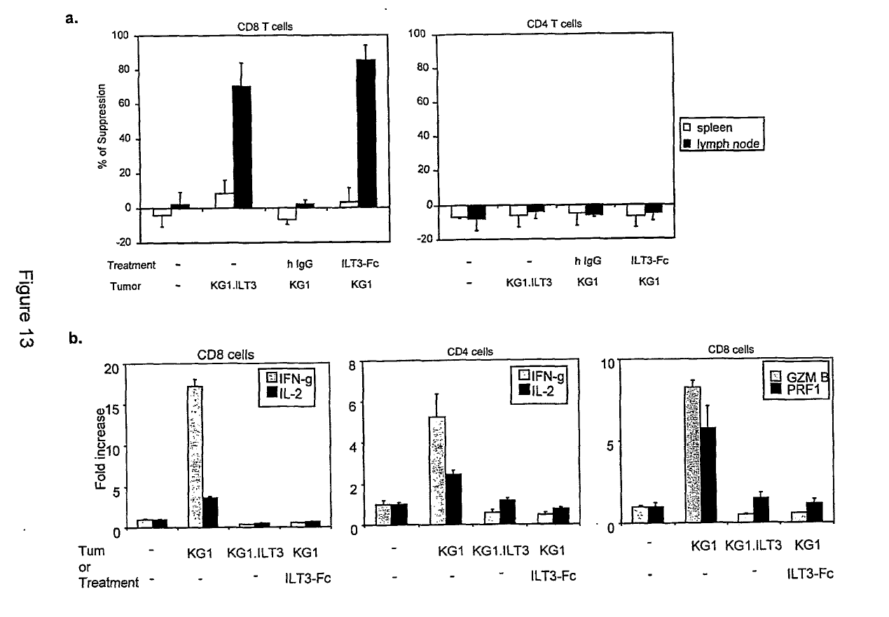

Human CD8⁺ T cells from lymph nodes of hu-SCID mice treated with sILT3 and transplanted with wild-type KG1 as well as CD8⁺ T cells from hu-SCID mice transplanted with KG1.ILT3 inhibited T cell responses to KG1 by >80% at a 4 to 1 ratio of T suppressor to T effector cells. CD8⁺ T cells from the same animals' spleen had no suppressor activity suggesting that they migrated from the tumor only to the draining lymph nodes or that they differentiated into Ts only in tumor-infiltrated lymph nodes. CD4⁺ T cells from the lymph nodes or spleen of these tolerant mice were not capable to inhibit the MLC response to KG1. CD4 and CD8 T cells from the lymph nodes and spleen of hu-SCID mice without tumor transplants or from IgG-treated controls which rejected the KG1 tumor transplant, showed no suppressor activity (FIG. 13A).

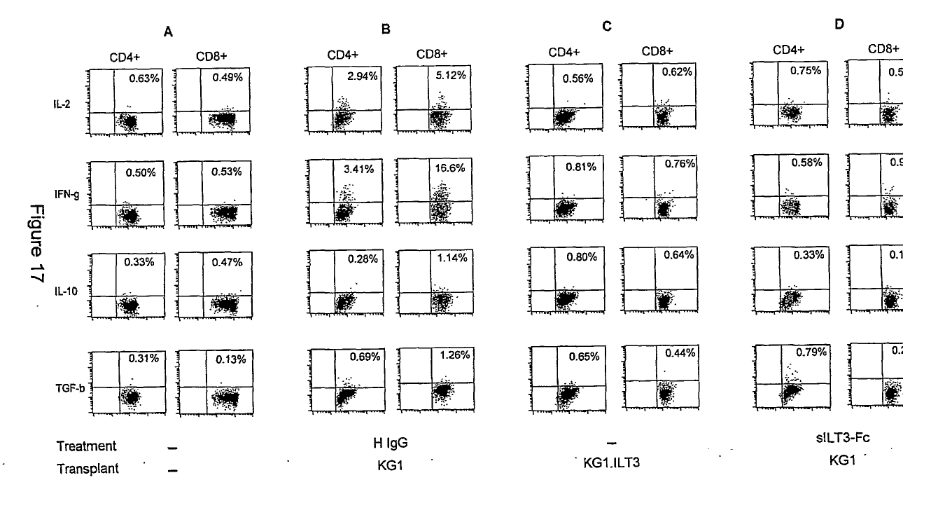

To further characterize human T cells from transplant draining lymph nodes their cytokine profile was determined by flow cytometry and quantitative RT-PCR (qRT-PCR). CD8⁺ T cells were also tested by qRT-PCR for granzyme B and perforin expression. The results obtained with both methods showed induction of IFN-γ and IL-2 in mice rejecting their tumor transplants but not in mice with growing tumors. No IL-10 or TGF-β producing T cells were detected in lymph nodes of any group of mice. CD8⁺ T cells from mice rejecting the tumor showed much higher levels of granzyme B and perforin compared to tolerant, tumor-bearing mice, exposed to ILT3 (FIGS. 13B and 17).

Sera from Patients with Cancer Contain Soluble ILT3

The finding that sILT3 induces tolerance to allogeneic human tumors in hu-SCID mice and that it promotes the differentiation of CD8⁺ T suppressor cells led to the idea that, if present in the circulation of patients with cancer, sILT3 may also abolish T cell responses against tumor antigens.

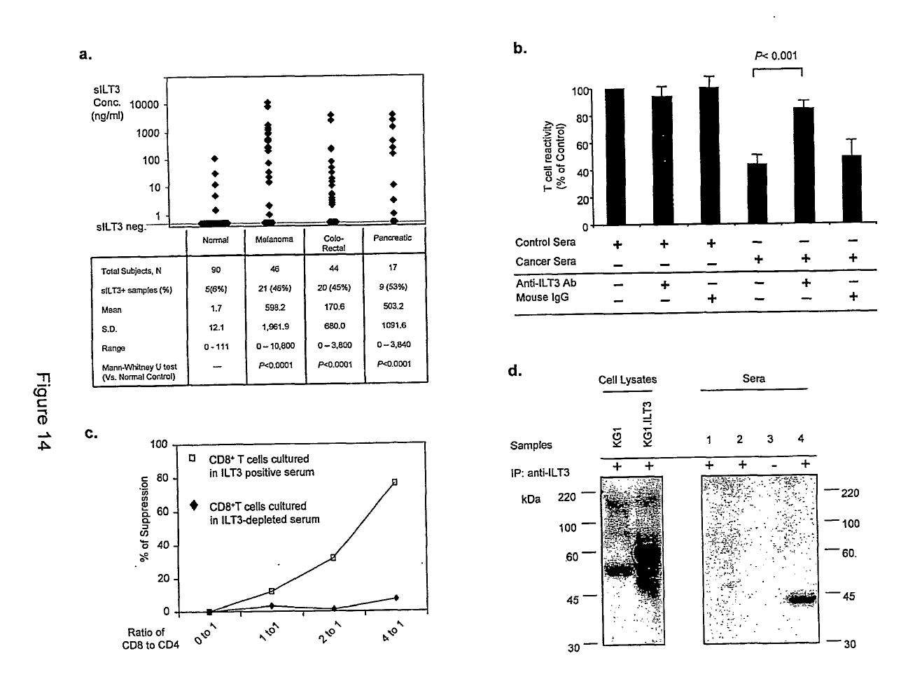

Sandwich ELISA studies of sILT3 showed that only 6% of healthy blood donors showed sILT3 while more than 40% of patients with melanoma, colorectal or pancreatic carcinoma had ILT3 in their sera. There was also a significantly higher amount of sILT3 in patients compared to controls. (FIG. 14A). The amount of sILT3 appeared to be stage-related in patients with colorectal carcinoma.

A cohort of 40 patients with hamatologic malignancy was also studied, and it was found that sILT3 can be of diagnostic value. Some patients showing no leukemic cells by flow cytometry or immunohistochemistry, did have 100-500 ng of ILT3 per ml serum. Further examination of such patients indicated a subset had cutaneous T cell lymphoma, thereby validating the diagnostic potential of sILT3 levels for certain cancers.

The inhibitory activity of ILT3 containing sera was assessed from 20 patients with colorectal and 9 with pancreatic carcinoma on the reactivity of T cells from healthy individuals in MLC performed in the presence or absence of murine mAb to ILT3 or of control mouse IgG.

Sera from cancer patients showed strong MLC-inhibitory activity which was abolished, however, by anti-ILT3 mAb. This indicates that sILT3 is responsible, at least in part, for the capacity of sera from cancer patients to inhibit T cell reactivity in vitro (FIG. 14B).

sILT3 Induces the Differentiation of CD8⁺ T_{S} Cells

To explore the possibility that serum ILT3 may induce the in vitro differentiation of CD8⁺ Ts CD3⁺CD25⁻ T cells from healthy responders with allogeneic APC were stimulated. CD8⁺ T cells, magnetically sorted from these cultures after 7 days, were added to MLC containing unprimed CD4⁺ CD25⁻ T cells from the same responder and APC from the original simulator. CD8⁺ T cells primed in the presence of serum containing sILT3 induced dose-dependent inhibition of T cell proliferation, while CD8⁺ T cells primed in ILT3-depleted serum had no Ts activity (FIG. 14C). ELISA and Western Blot Analysis confirmed the presence of sILT3 in serum prior to depletion and the removal of sILT3 after passage over sepharose beads coated with anti-ILT3 mAbs (FIG. 14D). The molecular weight of serum ILT3 was of approximately 45 kd. Taken together these data demonstrate that ILT3 contained by cancer patients' sera inhibits normal T cell reactivity to allogeneic APC and induces the differentiation of T_{S}.

### Expression of ILT3 in Tumors

Immunohistochemical studies of specimens from patients with melanoma demonstrated no ILT3 staining of tumors cells. However, strong membranous and cytoplasmic ILT3 staining was seen in cells surrounding the tumor. These cells may be of myeloid/histocytic origin as they co-expressed cell surface CD68, a macrophage marker.

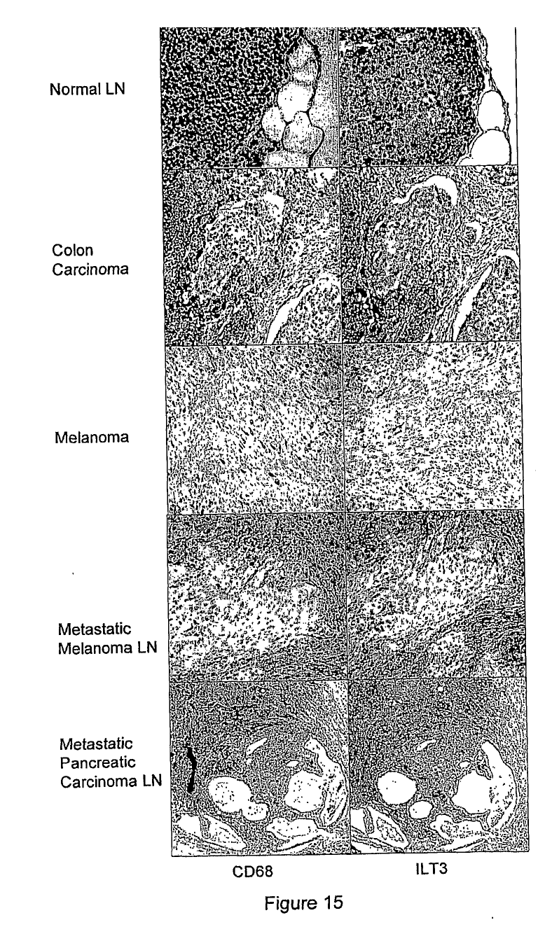

Studies of biopsy specimens from patients with adenocarcinoma of the pancreas also revealed strong membranous ILT3 staining of CD68⁺ macrophages. Examination of adjacent lymph nodes with no tumor cell infiltrates showed only few, weekly staining histiocytes. In sharp contrast, lymph nodes containing metastatic carcinoma demonstrated peri-tumoral infiltration by numerous histiocytes showing intense membranous staining for ILT3. Similar observations emerged from the examination of colorectal carcinoma (FIG. 15).

Alternatively Spliced ILT3 mRNAs

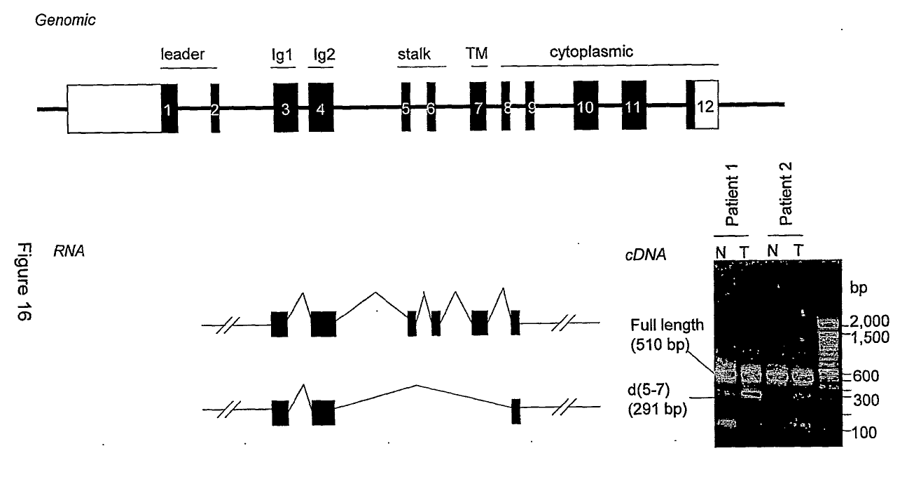

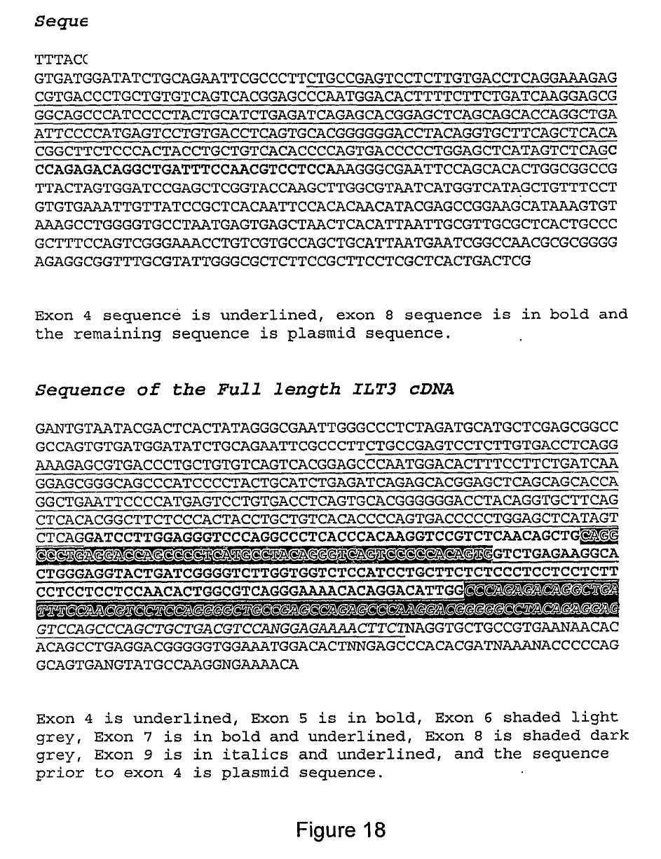

To test the possibility that soluble ILT3 may be transcribed by alternatively spliced mRNA, ILT3 specific primers were designed which can amplify mRNA from either exon 3 (first Ig-like domain) or exon 4 (second Ig-like domain) to exon 7 (transmembrane domain) or exon 8 (cytoplasmic domain) for RT-PCR analyses. Sequencing of amplified DNA obtained from frozen colon carcinoma biopsies showed deletion of exon 5 to 7 in 9 out of 13 cases (FIG. 16). Supernatants of CHO—S cell culture transfected with these splicing variants contained sILT3 which displayed the same molecular weight (45 kd) as that detected by Western Blot Analysis of cancer patients' sera. The full length ILT3 sequence (FIG. 18) showing each of the exons is provided, thereby elucidating the sILT3 sequence.

### Discussion

There is an emerging recognition that tumor growth elicits specific immune responses mediated by CD8⁺ and CD4⁺ T cells that may delay progression and be harnessed to eradicate malignant disease.(10)

T cell based immunotherapy has recently attracted much attention largely due to its success in experimental animal models as well as to the identification of tumor associated antigens (TAA) that might be suitable for immunotherapy in certain malignancies.(9,21) Many innovative approaches have focused on the activation of CD4⁺ Th capable to provide the help required for the growth and differentiation of tumor specific cytotoxic T cells. Because activated DC are highly efficient in stimulating immune responses numerous clinical studies have used DC loaded in various ways with TAA (DC vaccines) to induce CD8 and CD4 T cell responses against complexes formed by tumor specific peptides with MHC class I and class II molecules.(22) However, most immunotherapy trials have met with limited success, failing to demonstrate significant clinical responses.(22, 23) Combination immunotherapy such as adoptive transfer of in vivo primed T cells and post-transplant vaccination may foster enhanced memory T cell responses.(24,25)

Mechanisms responsible for tumor escape from immunosurveillance may include dysfunction of Th and Tc, expansion of CD4⁺ CD25⁺ regulatory T cells loss or down-regulation of HLA class I antigens or tumor-specific differentiation antigens, defective signaling through death receptor ligands such as FASL and TRAIL, lack of appropriate costimulation and production of immunosuppressive cytokines.(26,29)

In this study, the inhibitory effect of serum and membrane ILT3 are described, which may represent an additional mechanism that contributes to impaired T cell responses in patients with cancer. Using a hu-SCID mouse model, it was found that rILT3-Fc protein and membrane ILT3 inhibits T cell mediated rejection of human tumor allografts and induce the differentiation of allospecific CD8⁺ T suppressor cells.

However, CD4⁺ T cells from the same tumor bearing hu-SCID mouse had no regulatory activity. This finding is consistent with previous in vitro studies in which it was found that CD4⁺ T cells allostimulated in the presence of soluble or membrane ILT3 had no Tr activity, yet because they became anergic, they were unable to provide the help required for functional differentiation of IFN-γ producing CD8⁺ Tc (17). Instead, alloactivated CD8⁺ T cells from these cultures differentiated into Ts, which acted in an allorestricted manner on priming APC inducing them to upregulate the inhibitory ILT3 receptor.(17)

Since soluble ILT3 inhibits the rejection of allogeneic human tumors, it is apparent that if present in sera from patients with cancer it may have a similar effect on autologous tumors even if these express TAA.

The inhibitory activity of sera from cancer patients on the reactivity of autologous T cells or of allogeneic T cells from healthy individuals has long been known. (30,31) Elevated serum levels of IL-10 (an ILT3-inducer) were shown to correlate with poor clinical outcome.(32) More recently there has been increasing evidence that sera from patients with malignant disease contain soluble forms of NKG2D ligands which can potentially impair NKG2D-mediated immune function by blocking NKG2D receptors on NK and T cells.(33-35)

These studies herein show that soluble ILT3, present in a relatively high percentage of patients with various malignancies, inhibited strongly T cell responses in MLC. This inhibitory effect was partially abrogated by anti-ILT3 mAb indicating that it was caused by soluble ILT3. Furthermore, T cell allostimulation in cultures containing sILT3⁺ sera from cancer patients resulted in the differentiation of allospecific CD8⁺ T suppressor cells, consistent with the results obtained in the hu-SCID mouse model.

The main obstacle tempering successful immunotherapy and active vaccination may reside in the immunosuppressive effect displayed by regulatory CD4⁺CD25⁺ T cells which provide a crucial tumor evasion mechanism.(3,18,36) Stage related increases in the frequencies of CD4⁺CD25⁺ regulatory T cells were reported in numerous malignancies.(21)

CD8⁺ T suppressors that act in an antigen specific manner inhibiting the T cell priming capacity of the APC with which they interact or display their regulatory activity by producing IL-10, (similar to CD4⁺CD25⁺ Tr1 cells) have been described both in human and in rodents. (11-12, 37-42) Their contribution to tumor escape from immunosurveillance, however, has received less attention although the high frequency of non-cytolytic TAA specific CD8⁺ T cells found in patients with metastatic melanoma may reflect the presence of Ts rather than of Tc effector cells.(43,44)

The results herein indicate that membrane and soluble ILT3 may promote the differentiation of CD8⁺ Ts within the tumor microenvironment or in sentinel lymph nodes. Intensive membrane ILT3 staining of tumor associated CD68⁺ macrophages in colorectal and pancreatic carcinoma as well as in melanoma was found. The frequency of ILT3 expressing macrophage in tumor infiltrated lymph nodes was much higher than that seen in normal lymph nodes.

There is increasing evidence that tumor-associated macrophages (TAM) activated by immunosuppressors and IL-10 play an important role in tumor progression and metastasis.(45) TAMs were shown to possess poor antigen-presenting capacity, suppress T cell activation, and secrete a wide range of growth and proangiogenic factors as well as metalloproteinases. It has been suggested that cytokines present in the tumor microenvironment have the potential to induce the differentiation of recruited macrophages into TAMs which, in turn, produce growth factors and extracellular matrix enzymes facilitating tumor proliferation and invasion of surrounding tissue.(46,47) Recently a population of TAMs, characterized by B7-H4 expression and capacity to suppress TAA-specific T cell immunity, was identified in human ovarian carcinoma.(48)

Different mechanisms may account for the presence of soluble ILT3 in patients' circulation. By analogy to NKG2D ligands it is possible that sILT3 production is associated with post-translational proteolytic cleavage.(33,49) An alternative, not mutually exclusive mechanism supported by this study is that sILT3 found in patients' sera is secreted by TAM expressing alternative splice variants of ILT3 which lack the transmembrane domain, as also shown to be the case for other immunoregulatory receptors.(50) Ectopic expression of ILT3, as recently found in chronic lymphocytic leukemia may render tumor cells tolerogenic and provide an alternative source of both m and sILT3 (Adriana Colovai et al., in press).

Whatever the mechanism of sILT3 production, the data herein suggest that neither tumor vaccines nor adoptive therapy with TAA specific T cells are likely to be successful in patients with high levels of ILT3. Plasmapheresis with immunoabsorption of serum ILT3 may be necessary as a preliminary step before active or passive (adoptive) immunotherapy is initiated in patients with cancer. Identifying and blocking the ligand of ILT3 on activated T cells (17) may offer new strategies to enhance T cell immunity in cancer.

### Methods

Human subjects. Studied previously untreated patients with colorectal (N=44) and pancreas (N=17) adenocarcinoma. A cohort of patients with advanced stage melanoma (N=46) enrolled for treatment with high IL-2 doses were also included. The patients ranged in age from 20-68 years. Serum samples from healthy age matched blood donors (N=90) were used as controls. Buffy coats obtained from healthy blood donor volunteers were used for injection into SCID mice.

Human tumor cell lines. The myelomonocytic KG1, melanoma SK-ME-28 and pancreatic carcinoma PANC-1 lines were obtained from ATCC. KG1 tumor cell transfected with ILT3 (KG1.ILT3) or with the empty vector alone (KG1.MIG) were generated. Also used were the melanoma RE280 and T567A cell lines. Cell lines were maintained in RPMI supplemented with 10% heat-inactivated fetal calf serum.

Animals. C.B-17 SCID female mice were used at 5-8 wk of age. The animals were housed individually in micro isolator cages and were fed autoclaved food and water. Serum IgG levels were determined by sandwich ELISA using reagents from Cappel as previously described. SCID animals were considered “leaky” at IgG levels of >1 μg/ml and excluded from experimental use.

Generation, transplantation and treatment of hu-SCID mice. Human PBMC were isolated from fresh peripheral blood by Ficoll Hypaque centrifugation. SCID mice were reconstituted with 3×10⁸ human PBMC by i.p. inoculation and will be referred to as “hu-SCID” mice. Animals demonstrating signs of graft-vs-host disease or failing to reconstitute with human T cells were excluded from analysis by prior design. Circulating human T cells were evaluated by flow cytometry. Heparinized retro-orbital venous samples were obtained 2, 4, and 8 weeks after reconstitution, the erythrocytes were lysed and the phenotype of circulating human leukocytes was determined.

Concomitant with the humanizing treatment, mice were injected subcutaneously (s.c.) on the right flank with 2×10⁶ tumor cells. The tumor cell lines used for transplantation were the KG1, KG1.MIG, KG1.ILT3, SK-ME-28, RE280 and T567A. Groups of mice transplanted with “wild-type” KG1 or other human tumor cell lines were treated by i.p. injection of ILT3-Fc (250 μg/day) for the first 10 days following tumor and PBMC injection. Control hu-SCID mice received daily doses (250 μg) of human IgG (Sigma, St. Louis, Mo.) or were left untreated. Each experimental group consisted of 10 hu-SCID mice. Tumor growth was recorded every 3 days. For flow cytometry, immunohistochemistry and molecular studies additional SCID mice were “humanized” by the same method, transplanted, treated and sacrificed after 8 weeks.

Histology and immunohistochemistry. KG1 and KG1.ILT3 tumors growing in hu-SCID mice were harvested after 8 weeks and processed for paraffin-embedded sections. Immunostaining was performed using mouse anti-human CD3, CD4, CD8, CD68 and goat polyclonal Ab anti-human ILT3 (R&D Systems, Minneapolis, Minn.) or isotype-matched, nonbinding control Abs.

Frozen and fixed biopsies from the patients were examined for CD68 and ILT3 expression. Slides containing paraffin sections were dried in the oven overnight at 60 degrees C. Deparaffinization was accomplished using Pro-Par (xylene substitute) five times. The sections are then rehydrated in a series of alcohol dilutions (100%, 95%, 75% and 50%), then washed thoroughly in running warm water, followed by steaming during antigen retrieval for 40 minutes in pre-warmed Target Retrieval Solution (DakoCytomation, Carpenteria, Calif.). Sections were cooled for 10 minutes, stained on the Dako autostainer, then blocked for endogenous activity for 10 minutes in 3% hydrogen peroxide followed by incubation in goat anti-human ILT3 antibody at a 1:200 dilution for 40 minutes. After washing in TBS/20% Tween 20 they were incubated with secondary rabbit anti-goat antibody at a 1:200 dilution for 30 minutes. Next, sections were washed, blocked with 10% rabbit serum for 5 minutes and washed again. Slides were then incubated, utilizing the Vectastain Elite ABC kit (Vector Laboratories, Burlingame, Calif.) for 30 minutes; then washed and visualized with DAB+ (DakoCytomation, Carpenteria, Calif.)) for 5 minutes. The slides were then dehydrated and coverslipped using an automated coverslipper (Surgipath, Richmond, Ill.).

FACS analysis. Flow cytometry studies were performed on a FACSCalibur instrument using five-parameter acquisition (forward scatter, side scatter, and three fluorescence channels) (BD Biosciences). The following mAbs were used: anti-HLA-ABC-FITC, HLA-DR-FITC, CD4-FITC, CD8-FITC, CD86-FITC, CD11c-PE, CD25-PE, IL-2-PE, IFN-gamma-PE, IL-10-PE, TGF-beta-PE, CD3-CyChrome, (all from BD Biosciences), and ILT3-PC5 (Coulter). For cytokine studies, purified CD4 and CD8 cells were activated for 6 hours on CD3 antibody coated plates (BD) in the presence of 1 ug/ml of CD28 mAb (BD). Brefeldin A (1 ug/ml) was added to the culture for the last 3 hours of incubation. Cells were first stained with surface markers, then for intracellular cytokine expression using the Cytofix/Cytoperm kit (BD) according to the manufacturer's instructions. For each marker, a corresponding isotype-matched Ab conjugated with the same fluorescent dye was used as a negative control. The percent of human CD4 an CD8 T cells present in the peripheral blood, spleen, axillary and inguinal draining lymph nodes was determined.

T suppressor cell assays. Human CD4⁺ and CD8⁺ T cells were sorted from lymph nodes and spleen of hu-SCID mice at 8 weeks using the CD4 or CD8-isolation kits (Stem Cell Technology, Vancouver, Canada). Increasing numbers of sorted CD4⁺ or CD8⁺ cells were added to a fixed number (10⁴) of unprimed autologous CD3⁺CD25⁻ T cells and stimulated for 6 days in MLC with irradiated KG1 cells.

To assay the T suppressor activity of human T cells primed in the presence of sera from cancer patients we primed CD3⁺CD25⁻ T cells isolated from fresh peripheral blood of healthy volunteers with irradiated APC from an allogeneic donor differing by two HLA-DR antigens from the responder. Cultures were performed in the presence of sILT3 positive or sILT3-depleted sera from patients with carcinoma of the pancreas and colon. CD8⁺ T cells, magnetically sorted from these cultures after 7 days, were added to MLC containing unprimed CD4⁺CD25⁻ T cells from the same responder and APC from the original simulator. [³H] Thymidine was added to the cultures 18 h before harvesting and incorporation was determined by scintillation spectrometry using a LKB 1250 Betaplate counter (Perkin Elmer, Wellesley, Mass.). Mean cpm of triplicate cultures and the SD from the mean were calculated. Percentage suppression was calculated according to the formula [1−CPM(Th+Ts vs KG1)/CPM(Th vs KG1)]×100.

Sandwich ELISA detection of serum ILT3. High-binding 96-well “Maxisorp” plates (Nalge Nunc International, Rochester, N.Y.) were coated overnight at 4° C. with 1.0 μg/well of anti-ILT3 mAb (clone ZL5.7, developed in our laboratory) then free binding sites were blocked with 5% BSA solution for 1 hour at room temperature. After washing with PBS-T (PBS+0.1% Tween 20), 100 μl serum samples were added to the wells in 1:3 and 1:9 dilutions. After 1 hour of incubation, the plate was washed and 100 μl of anti-ILT3 biotinylated polyclonal Ab (R&D Systems) was added at a concentration of 33 ng/ml to each well followed by 1-hour incubation. After washing, 100 μl of 1 ug/ml horseradish peroxidase (HRP)-conjugated streptavidin (BD Biosciences) was added to each well and incubated for 1 hour. After washing, the plate was developed using 100 μl of tetramethylbenzidine (TMB) substrate (Sigma-Aldrich). Reactions were stopped using 100 μl/well of 1N H₂SO₄, and the plates were read at 450 nm. For calibration of each sandwich ELISA, standards in the range of 0-1500 ng/ml rILT3-Fc diluted in 10% FCS were run on each plate with the test samples.

Immunoprecipiation and Western Blotting Assays were conducted as previously described (ref. 17). Briefly, KG1 and KG1.ILT3 cells (1×10⁶) were homogenized and (1 ml) aliquots of serum samples previously tested by ELISA for presence of sILT3 were thawed before testing. All samples were vigorously centrifuged and pre-cleared with 0.1 volumes of (packed) Agarose-Protein G beads (Millipore, Billerica, Mass.). Immunoprecipitation of ILT3 was carried out using the same Agarose-Protein G beads as used for pre-clearing plus 5 μg/ml mAb anti-ILT3 (clone ZM3.8). Western blotting was conducted using a biotinylated anti-ILT3 detection antibody (R&D Systems) and the ECL detection system.

Serum inhibition studies. PBMC from healthy blood donors were separated from buffy coats by density gradient centrifugation. CD3+ T cells were obtained using the T cell isolation kits (Miltenyi Biotec, Auburn, Calif.) according to the manufacturer's instructions. CD25⁺ T cells were depleted by use of CD25 beads (Miltenyi Biotech) from CD3⁺ T cells tested as responders in functional assays. All cell cultures were performed in complete medium (RPMI 1640 supplemented with 2 mM L-glutamine, and 50 mg/L gentamicin from Mediatech).

CD3⁺CD25⁻ T cells from healthy responders (5×10⁴/well) were stimulated in 6-day MLC with irradiated (5000 rads) KG1 cells (2.5×10⁴/well). Reactivity in normal serum was established by adding to 12 replicate wells 20% pooled serum from healthy blood donors. Sera from individual patients with colorectal or pancreatic cancer, in which sILT3 was detected, were added to triplicate reactions at a final concentration of 20%. Serum from the same patient was tested in parallel triplicate cultures to which 5 μg/ml of mAb anti-ILT3 or 5 μg of mouse IgG were added.

T cell reactivity in pooled normal serum was normalized to 100%. Reactivity of the same responder's T cells in cultures containing sera from individual cancer patients was expressed as percent of reactivity seen in normal serum. Reactivity in patient serum supplemented with anti-ILT3 mAb or mouse IgG control was calculated in the same manner.

Quantitative real-time polymerase chain reaction (PCR). Total RNA was isolated from human cells using the RNAqueous Kit (Ambion, Austin Tex.), and reverse-transcribed using the First Strand cDNA Synthesis Kit for RT-PCR (Roche Diagnostics, Indianapolis, Ind.). Quantitative real-time PCR was performed using Taqman gene expression assays (Applied Biosystems, Foster City, Calif.). The gene expression assays used in these studies are identified below, by gene name, manufacturer's abbreviation and part number.

| Gene name | AB Abbrev | AB Assay ID |
|---|---|---|
| GAPDH | GAPDH | 4326317E |
| Granzyme B | GZMB | Hs00188051_m1 |
| Perforin | PRF1 | Hs00169473_m1 |
| Interleukin 2 | IL2 | Hs00174114_m1 |
| Interferon gamma | IFNG | Hs00174143_m1 |
| Interleukin 10 | IL10 | Hs00174086_m1 |

The assay primers share the following general features: (a) forward and reverse primers are contained in different exons, when multiple exons encode the transcript; (b) the reporter probe spans the exon-exon junction of the targeted transcript to prevent amplification of genomic DNA; and (c) sense and antisense primers are typically located ≈25-50 bp on either side of the exon-exon junction.

The 7300 RT-PCR instrument (Applied Biosystems) may be used, applying the manufacturer's protocols and recommended amplification conditions, as follows: one cycle at 50° C. (2 min), then 95° C. (10 min), followed by 50 cycles at 95° C. (15 s) and 60° C. (1 min). Data were collected and analyzed using the 7300 SDS 1.3.1 software (Applied Biosystems). Each assay plate included “no template” negative controls and a control cDNA. The relative amount of gene expression was calculated according to the formula: 2^{-ΔCt}, where ΔCt=[Ct(gene)−Ct(GAPDH)].

Isolation and cloning of alternatively spliced ILT3 transcripts. Total RNA was isolated from frozen biopsy tissue obtained from 13 patients with colon adenocarcinoma. Both normal and malignant tissue from the same individual was assayed. For amplification of ILT3 specific cDNA 1-3 μL of individual RNA preparations were added to 25 μL of One step—RT-PCR (SuperScript® One-Step RT-PCR, Invitrogen, Calif.) reactions together with ILT3 exon (3rd-8th) specific primers. The following primers were synthesized by a commercial vendor (Invitrogen, Carlsbad, Calif.). Exon 3 Forward: 5′-CCC CTC CCC AAA CCC ACC CTC-3′; Exon 4 Forward: 5′-CTG CCG GTC CTC TTG TGA CC-3′; Exon 7 Reverse 5′-AGA GGA GGA GGG AGA GAA GCA GGA TG-3′ and Exon 8 Reverse 5′-TGG AGG ACG TTG GAA ATC AGC-3′. The thermal profiles typically consisted of 1 cycle of 50° C. 30′ and 94° C. 2′ followed by 40 cycles of 94° C. 20″, 60° C. 30″, 72° C. 30″ and one cycle of 72° C. 7′. PCR amplification products were fractionated by 1.3% agarose gel electrophoresis. DNA with variant sizes were purified, cloned into a TA cloning vector using TOPO cloning kit (Invitrogen) and completely sequenced from both strands.

For expression of spliced ILT3 mRNA obtained from colon carcinoma tissue, we first cloned the spliced cDNA using the exon 4 primer described above and a primer corresponding to exon 11 (5′-ATC GAA TTC GGA TTA GTG GAT GGC CAG AG-3′) which includes the stop codon of ILT3. Amplified products were digested with EcoRI and 410 bp fragments containing alternatively spliced variants of exon 4-8 were used to replace the 600 bp EcoRI fragment of the ILT3 sequence previously cloned by us (12, 17). The splice variant has expressed in CHO—S cells as previously described (17). The supernatant was collected and tested by ELISA and Western Blot Analysis using anti-ILT3 antibodies as described above.

Statistics. ELISA data was analyzed using nonparametric Mann-Whitney U-test to compare sILT3 in sera from healthy individuals and patients with cancer. The statistical difference between T cell reactivity in serum from healthy individuals and patients with cancer in the presence or absence of anti-ILT3 mAb or mouse IgG was analyzed using paired two-tailed Student t-test. Calculations were performed using BMDP release 7 (BMDP Statistical Software, Los Angeles, Calif.).

### REFERENCES FOR SECOND SERIES OF EXPERIMENTS

1. Steinman et al. Avoiding horror autotoxicus: the importance of dendritic cells in peripheral T cell tolerance. Proc Natl Acad Sci USA 99, 351-8 (2002).
2. Lanzavecchia et al. Immunology. Licence to kill. Nature 393, 413-4 (1998).
3. Vlad et al. License to heal: bidirectional interaction of antigen-specific regulatory T cells and tolerogenic APC. J Immunol 174, 5907-14 (2005).
4. Bluestone et al. Natural versus adaptive regulatory T cells. Nat Rev Immunol 3, 253-7 (2003).
5. Sakaguchi et al. Naturally arising CD4+ regulatory t cells for immunologic self-tolerance and negative control of immune responses. Annu Rev Immunol 22, 531-62 (2004).
6. Shevach, E. M. CD4+ CD25+ suppressor T cells: more questions than answers. Nat Rev Immunol 2, 389-400 (2002).
7. Fontenot et al., Foxp3 programs the development and function of CD4+CD25+ regulatory T cells. Nat Immunol 4, 330-6 (2003).
8. Khattri et al. An essential role for Scurfin in CD4+CD25+ T regulatory cells. Nat Immunol 4, 337-42 (2003).
9. Zou, W. Regulatory T cells, tumour immunity and immunotherapy. Nat Rev Immunol 6, 295-307 (2006).
10. Khazaie et al. The impact of CD4+CD25+ Treg on tumor specific CD8+ T cell cytotoxicity and cancer. Semin Cancer Biol 16, 124-36 (2006).
11. Liu et al. Effects of pregnant mare serum gonadotropin (eCG) on follicle development and granulosa-cell apoptosis in the pig. Theriogenology 59, 775-85 (2003).
12. Chang et al. Tolerization of dendritic cells by T(S) cells: the crucial role of inhibitory receptors ILT3 and ILT4. Nat Immunol 3, 237-43 (2002).
13. Manavalan et al. High expression of ILT3 and ILT4 is a general feature of tolerogenic dendritic cells. Transpl Immunol 11, 245-58 (2003).
14. Cella et al. A novel inhibitory receptor (ILT3) expressed on monocytes, macrophages, and dendritic cells involved in antigen processing. J Exp Med 185, 1743-51 (1997).
15. Colonna et al. Human myelomonocytic cells express an inhibitory receptor for classical and nonclassical MHC class 1 molecules. J Immunol 160, 3096-100 (1998).
16. Colonna et al. A family of inhibitory and activating Ig-like receptors that modulate function of lymphoid and myeloid cells. Semin Immunol 12, 121-7 (2000).
17. Kim-Schulze et al. Recombinant Ig-like transcript 3-Fc modulates T cell responses via induction of Th anergy and differentiation of CD8+ T suppressor cells. J Immunol 176, 2790-8 (2006).
18. Shevach, E. M. Fatal attraction: tumors beckon regulatory T cells. Nat Med 10, 900-1 (2004).
19. Curiel et al. Specific recruitment of regulatory T cells in ovarian carcinoma fosters immune privilege and predicts reduced survival. Nat Med 10, 942-9 (2004).
20. Ghiringhelli et al. CD4+CD25+ regulatory T cells inhibit natural killer cell functions in a transforming growth factor-beta-dependent manner. J Exp Med 202, 1075-85 (2005).
21. Beyer et al. Regulatory T cells in cancer. Blood 108, 804-11 (2006).
22. Figdor et al. Dendritic cell immunotherapy: mapping the way. Nat Med 10, 475-80 (2004).
23. Shu et al. Immune responses in the draining lymph nodes against cancer: implications for immunotherapy. Cancer Metastasis Rev 25, 233-42 (2006).
24. Rapoport et al. Restoration of immunity in lymphopenic individuals with cancer by vaccination and adoptive T-cell transfer. Nat Med 11, 1230-7 (2005).
25. Rosenberg et al. Cancer immunotherapy: moving beyond current vaccines. Nat Med 10, 909-15 (2004).
26. Khong et al. Natural selection of tumor variants in the generation of “tumor escape” phenotypes. Nat Immunol 3, 999-1005 (2002).
27. Ferris et al. Immune escape associated with functional defects in antigen-processing machinery in head and neck cancer. Clin Cancer Res 12, 3890-5 (2006).
28. Dunn et al. The immunobiology of cancer immunosurveillance and immunoediting. Immunity 21, 137-48 (2004).
29. Pinzon-Charry et al. Dendritic cell dysfunction in cancer: a mechanism for immunosuppression. Immunol Cell Biol 83, 451-61 (2005).
30. Suciu-Foca et al. Impaired responsiveness of lymphocytes and serum-inhibitory factors in patients with cancer. Cancer Res 33, 2373-7 (1973).
31. Wanebo et al. Contribution of serum inhibitory factors and immune cellular defects to the depressed cell-mediated immunity in patients with head and neck cancer. Am J Surg 166, 389-94 (1993).
32. Bohlen et al. Poor clinical outcome of patients with Hodgkin's disease and elevated interleukin-10 serum levels. Clinical significance of interleukin-10 serum levels for Hodgkin's disease. Ann Hematol 79, 110-3 (2000).
33. Chang et al. NK cell activating ligands on human malignant cells: Molecular and functional defects and potential clinical relevance. Semin Cancer Biol 16, 383-92 (2006).
34. Coudert et al. The role of the NKG2D receptor for tumor immunity. Semin Cancer Biol 16, 333-43 (2006).
35. Groh et al. Fas-ligand-mediated paracrine T cell regulation by the receptor NKG2D in tumor immunity. Nat Immunol 7, 755-62 (2006).
36. Nomura et al. Naturally arising CD25+CD4+ regulatory T cells in tumor immunity. Curr Top Microbiol Immunol 293, 287-302 (2005).
37. Rifa'i et al. Essential roles of CD8+CD122+ regulatory T cells in the maintenance of T cell homeostasis. J Exp Med 200, 1123-34 (2004).
38. Gilliet et al. Generation of human CD8 T regulatory cells by CD40 ligand-activated plasmacytoid dendritic cells. J Exp Med 195, 695-704 (2002).
39. Colonna et al. Plasmacytoid dendritic cells in immunity. Nat Immunol 5, 1219-26 (2004).
40. Dhodapkar et al. Antigen-specific inhibition of effector T cell function in humans after injection of immature dendritic cells. J Exp Med 193, 233-8 (2001).
41. Steinbrink et al. CD4(+) and CD8(+) anergic T cells induced by interleukin-10-treated human dendritic cells display antigen-specific suppressor activity. Blood 99, 2468-76 (2002).
42. Kapp et al. TCR transgenic CD8+ T cells activated in the presence of TGF{beta} express FoxP3 and mediate linked suppression of primary immune responses and cardiac allograft rejection. Int Immunol, Sep. 11 (2006).
43. Zippelius et al. Effector function of human tumor-specific CD8 T cells in melanoma lesions: a state of local functional tolerance. Cancer Res 64, 2865-73 (2004).
44. Lee et al. Characterization of circulating T cells specific for tumor-associated antigens in melanoma patients. Nat Med 5, 677-85 (1999).
45. Luo et al. Targeting tumor-associated macrophages as a novel strategy against breast cancer. J Clin Invest 116, 2132-2141 (2006).
46. Mantovani et al. Macrophage polarization: tumor-associated macrophages as a paradigm for polarized M2 mononuclear phagocytes. Trends Immunol 23, 549-55 (2002).
47. Gordon, S. Alternative activation of macrophages. Nat Rev Immunol 3, 23-35 (2003).
48. Kryczek et al. B7-H4 expression identifies a novel suppressive macrophage population in human ovarian carcinoma. J Exp Med 203, 871-81 (2006).
49. Waldhauer et al. Steinle, A. Proteolytic release of soluble UL16-binding protein 2 from tumor cells. Cancer Res 66, 2520-6 (2006).
50. Nielsen et al. Alternative splice variants of the human PD-1 gene. Cell Immunol 235, 109-116 (2006).

What is claimed is:

1. A method of treating a subject afflicted with a disease selected from chronic viral disease and cancer, comprising withdrawing blood from the subject, removing sILT3 from the withdrawn blood, and returning the sILT3-depleted blood to the subject, thereby treating the subject afflicted with the disease.

2. The method of claim 1, wherein the disease is cancer, and the treatment results in cancer remission.

3. The method of claim 1, wherein the disease is chronic viral disease, and the treatment results in an enhancement of the subject's ability to mount an immune response.

4. A method of enhancing a subject's ability to mount an immune response, comprising withdrawing blood from the subject, removing sILT3 from the withdrawn blood, and returning the sILT3-depleted blood to the subject, thereby enhancing the subject's ability to mount an immune response.

5. The method of claim 4 wherein the subject suffers from a disease characterized by increased levels of sILT3 in blood.

6. The method of claim 4 wherein the subject suffers from chronic viral disease or cancer.

7. A method of enhancing remission of a tumor, comprising withdrawing blood from the subject, removing sILT3 from the withdrawn blood, and returning the sILT3-depleted blood to the subject, thereby enhancing remission of the tumor.

8. A method of decreasing tolerance to a tumor in a subject, comprising withdrawing blood from the subject, removing sILT3 from the withdrawn blood, and returning the sILT3-depleted blood to the subject, thereby decreasing tolerance to the tumor.

9. The method of any one of claims 1-8, further comprising administering an anti-ILT3 antibody to the subject.

10. A method of treating a subject afflicted with a disease selected from chronic viral disease and cancer, comprising administering to the subject an anti-ILT3 antibody, thereby treating the subject.

11. A method of enhancing a subject's ability to mount an immune response, comprising administering to the subject an anti-ILT3 antibody, thereby enhancing the subject's ability to mount an immune response.

12. A method of enhancing remission of a tumor, comprising administering to the subject an anti-ILT3 antibody, thereby enhancing remission of the tumor.

13. A method of decreasing tolerance to a tumor in a subject, comprising administering to the subject an anti-ILT3 antibody, thereby decreasing tolerance to the tumor.

14. A method of determining tumor metastasis comprising identifying the presence of ILT3+ cells in a sample from the tumor microenvironment or lymph node of a subject, wherein the presence of mILT3 indicates tumor metastasis in the subject.

15. The method of claim 14, wherein the lymph node is the sentinel lymph node.

16. The method of claim 14, wherein the cells are tumor associated macrophage cells.

17. The method of claim 16, wherein the cells are CD68⁺ cells.

18. The method of claim 17, wherein the subject is afflicted with melanoma, pancreatic carcinoma or colorectal carcinoma.

19. A method of diagnosing cancer in a subject comprising identifying the presence of sILT3 in the subject's serum, wherein the presence of sILT3 diagnoses cancer in the subject.

20. The method of claim 19, wherein the cancer is melanoma, colorectal carcinoma, pancreatic carcinoma, or hematologic malignancy.

21. The method of claim 20, wherein the cancer is hematologic malignancy, and the hematologic malignancy is cutaneous T cell lymphoma, chronic lymphocytic leukemia, acute lymphocytic leukemia or Hodgkin's Lymphoma.

22. The method of claim 19, wherein the sILT3 is present in a concentration of at least 100 ng/ml.

23. A method of determining the progression of cancer in a subject comprising determining the sILT3 concentration in the subject, and comparing the sILT3 concentration with a sILT3 concentration previously determined for the subject, wherein a higher concentration indicates that the cancer has progressed to a more advanced stage, and a lower concentration indicates that the cancer has regressed to a less advanced stage.

24. A method of treating a subject afflicted with a disease selected from chronic viral disease and cancer, comprising administering to the subject a composition comprising an RNAi construct targeted to an sILT3 mRNA, in an amount sufficient to attenuate expression of sILT3, thereby treating the subject.

25. A nucleic acid comprising a nucleotide sequence of a splice variant of ILT3 as shown in FIG. 18.
